(19) United States
(12) Patent Application Publication
(10) Pub. No.: US 2018/0265613 A1
(43) Pub. Date: Sep. 20, 2018

(54) SEQUESTRANTS OF ADVANCED GLYCATION END PRODUCT (AGE) PRECURSORS

(71) Applicant: GENZYME CORPORATION, Cambridge, MA (US)

(72) Inventors: Stephen Randall Holmes-Farley, Arlington, MA (US); Pradeep K. Dhal, Westford, MA (US); Magnus Besev, Newton, MA (US); Robert J. Miller, East Bridgewater, MA (US); Andrew T. Papoulis, Canton, MA (US)

(21) Appl. No.: 15/842,177

(22) Filed: Dec. 14, 2017

Related U.S. Application Data
(62) Division of application No. 14/776,059, filed on Sep. 14, 2015, now abandoned.
(60) Provisional application No. 61/792,719, filed on Mar. 15, 2013.

(51) Int. Cl. C08F 126/02; A61K 31/785

(52) U.S. Cl. CPC C08F 126/02; A61K 31/785

(57) ABSTRACT

Sequestrants of AGE precursors comprise amines separated by 2, 3 or 4 carbons. Sequestrants of AGE precursors can be used as pharmaceutical agents and in pharmaceutical compositions. The sequestrants of AGE precursors are particularly useful binding AGE precursors and dietary dicarbonyls in mammals in the gastrointestinal tract for the treatment of ailments such as diabetic nephropathy, chronic renal disease, atherosclerosis, stroke, cataracts, and Alzheimer's disease.

### CROSS-REFERENCE TO RELATED APPLICATIONS

Not applicable

### STATEMENT REGARDING FEDERALLY SPONSORED RESEARCH

Not applicable

### THE NAMES OF THE PARTIES TO A JOINT RESEARCH AGREEMENT

Not applicable

### INCORPORATION-BY-REFERENCE OF MATERIAL SUBMITTED ON COMPACT DISC

Not applicable

### BACKGROUND OF THE INVENTION

### Field of the Invention

This invention relates to sequestrants of advanced glycation end product (AGE) precursors. The sequestrants of AGE precursors bind dietary dicarbonyls, a key precursor in AGE formation. This invention further relates to the use of sequestrants of AGE precursors as pharmaceutical agents and in pharmaceutical compositions and to the use of sequestrants of AGE precursors to bind AGE precursors and dietary dicarbonyls.

### Definitions

As used herein, the term “amino” means a functional group having a nitrogen atom and 1 to 2 hydrogen atoms. “Amino” generally may be used herein to describe a primary, secondary, or tertiary amine, and those of skill in the art will readily be able to ascertain the identification of which in view of the context in which this term is used in the present disclosure. The term “amine” or “amine group” or “ammonia group” means a functional group containing a nitrogen atom derived from ammonia (NH₃). The amine groups are preferably primary amines, meaning the nitrogen is bonded to two hydrogen atoms and one substituent group comprising a substituted or unsubstituted alkyl or aryl group or an aliphatic or aromatic group. The amine groups may be secondary amines meaning, the nitrogen is bonded to one hydrogen atom and two substituent groups comprising a substituted or unsubstituted alkyl or aryl groups or an aliphatic or aromatic group, as defined below. The amine groups may be tertiary amines meaning the nitrogen is bonded to three substituent groups comprising a substituted or unsubstituted alkyl or aryl groups or an aliphatic or aromatic group. The amine groups may also be quaternary amines meaning the designated amine group is bonded to a fourth group, resulting in a positively charged ammonium group.

It is understood that any or all of the amines in the present invention may be in the free amine form (that is, as —NH₂ for a primary amine) or in a protonated form with a pharmaceutically acceptable anion (that is, as —NH₃⁺Y⁻ for a primary amine, where Y⁻ is the pharmaceutically acceptable anion).

As used herein, the term “amide group” means a functional group comprising a carbonyl group linked to a nitrogen. “Carbonyl” or a “carbonyl group” means a functional group comprising a carbon atom double bonded to an oxygen atom, represented by (C═O).

The term “alkane” means a saturated hydrocarbon, bonded by single bonds. Alkanes can be linear or branched. “Cycloalkanes” are saturated hydrocarbons rings bonded by single bonds.

As used herein, the term “(C₁-C₁₀)alkyl” means a saturated straight chained or branched or cyclic hydrocarbon consisting essentially of 1 to 10 carbon atoms and a corresponding number of hydrogen atoms. Typically straight chained or branched groups have from one to ten carbons, or more typically one to five carbons. Exemplary (C₁-C₁₀)alkyl groups include methyl (represented by —CH₃), ethyl (represented by —CH₂—CH₃), n-propyl, isopropyl, n-butyl, isobutyl, etc. Other (C₁-C-₁₀)alkyl groups will be readily apparent to those of skill in the art given the benefit of the present disclosure.

As used herein, the term “(C₂-C₉)heteroalkyl” means a saturated straight chained or branched or cyclic hydrocarbon consisting essentially of 2 to 10 atoms, wherein 2 to 9 of the atoms are carbon and the remaining atom(s) is selected from the group consisting of nitrogen, sulfur, and oxygen. Exemplary (C₂-C₉)heteroalkyl groups will be readily apparent to those of skill in the art given the benefit of the present disclosure.

As used herein, the term “(C₃-C₁₀)cycloalkyl” means a nonaromatic saturated hydrocarbon group, forming at least one ring consisting essential of 3 to 10 carbon atoms and a corresponding number of hydrogen atoms. (C₃-C₁₀)cycloalkyl groups can be monocyclic or multicyclic. Individual rings of multicyclic cycloalkyl groups can have different connectivities, for example, fused, bridged, spiro, etc., in addition to covalent bond substitution. Exemplary (C₃-C₁₀)cycloalkyl groups include cyclopropyl, cyclobutyl, cyclopentyl, cyclohexyl, norbornanyl, bicyclo-octanyl, octahydro-pentalenyl, spiro-decanyl, cyclopropyl substituted with cyclobutyl, cyclobutyl substituted with cyclopentyl, cyclohexyl substituted with cyclopropyl, etc. Other (C₃-C₁₀)cycloalkyl groups will be readily apparent to those of skill in the art given the benefit of the present disclosure.

As used herein, the term “(C₂-C₉)heterocycloalkyl” means a nonaromatic group having 3 to 10 atoms that form at least one ring, wherein 2 to 9 of the ring atoms are carbon and the remaining ring atom(s) is selected from the group consisting of nitrogen, sulfur, and oxygen. (C₂-C₉)heterocycloalkyl groups can be monocyclic or multicyclic. Individual rings of such multicyclic heterocycloalkyl groups can have different connectivities, for example, fused, bridged, spiro, etc., in addition to covalent bond substitution. Exemplary (C₂-C₉)heterocycloalkyl groups include pyrrolidinyl, tetrahydrofuranyl, dihydrofuranyl, tetrahydropyranyl, pyranyl, thiopyranyl, aziridinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl, 2-azaspiro[4.4]nonanyl, 7-oxa-1-aza-spiro[4.4]nonanyl, 7-azabicyclo[2.2.2]heptanyl, octahydro-1H-indolyl, etc. The (C₂-C₉)heterocycloalkyl group is typically attached to the main structure via a carbon atom or a nitrogen atom. Other (C₂-C₉)heterocycloalkyl groups will be readily apparent to those of skill in the art given the benefit of the present disclosure.

The term “aliphatic group” or “aliphatic” means a non-aromatic group consisting of carbon and hydrogen, and may optionally include one or more double and/or triple bonds. In other words, an aliphatic group is any group consisting of carbon and hydrogen which contains no aromatic functionality. An aliphatic group may be straight chained, branched or cyclic and typically contains between about one and about 24 carbon atoms.

The term “aryl group” may be used interchangeably with “aryl,” “aryl ring,” “aromatic,” “aromatic group,” and “aromatic ring.” Aryl groups include carbocyclic aromatic groups, typically with six to fourteen ring carbon atoms. Aryl groups also include heteroaryl groups, which typically have five to fourteen ring atoms with one or more heteroatoms selected from nitrogen, oxygen and sulfur.

As used herein, the term “(C₆-C₁₄)aryl” means an aromatic functional group having 6 to 14 carbon atoms that form at least one ring.

As used herein, the term “(C₂-C₉)heteroaryl” means an aromatic functional group having 5 to 10 atoms that form at least one ring, wherein 2 to 9 of the ring atoms are carbon and the remaining ring atom(s) is selected from the group consisting of nitrogen, sulfur, and oxygen. (C₂-C₉)heteroaryl groups can be monocyclic or multicyclic. Individual rings of such multicyclic heteroaryl groups can have different connectivities, for example, fused, etc., in addition to covalent bond substitution. Exemplary (C₂-C₉)heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. The (C₂-C₉)heteroaryl group is typically attached to the main structure via a carbon atom, however, those of skill in the art will realize when certain other atoms, for example, hetero ring atoms, can be attached to the main structure. Other (C₂-C₉)heteroaryl groups will be readily apparent to those of skill in the art given the benefit of the present disclosure.

As used herein, the term “alkyl amine” means an (C₁-C₁₀)alkyl containing a primary, secondary, or tertiary amine group in place of one hydrogen atom, represented by (C₁-C₁₀)alkyl amine and ((C₁-C₁₀)alkyl)₂ amine.

The term “alkyl ester” means a (C₁-C₁₀)alkyl containing an ester group in place of one hydrogen atom, represented by —O(O)C—(C₁-C₁₀)alkyl.

The term “alkyl acid” means an (C₁-C₁₀)alkyl containing a carboxylic acid group in place of one hydrogen atom, represented by (C₁-C₁₀)alkyl-COOH.

The term “aliphatic acid” means an acid of nonaromatic hydrocarbons, represented by (C₁-C₁₀)alkyl-COOH and (C₃-C₁₀)cycloalkyl-COOH.

The term “halo” means a fluorine (F), chlorine (Cl), bromine (Br), iodine (I), or astatine (At) ion.

The term “methoxy” means a (C₁)alkyl containing an oxygen in place of one hydrogen atom, represented by —(O)CH₃.

The term “polyol” means an alcohol containing multiple hydroxyl (—OH) groups.

“Substituted” means the substitution of a carbon in alkyl, heterocyclic or aryl groups with one or more non-carbon substituent. Non-carbon substituents are selected from nitrogen, oxygen and sulfur.

“Unsubstituted” means the group is comprised of only hydrogen and carbon.

The term “polymer” means a molecule with a molecular weight over 1,000 Daltons comprised of one or more repeat units. The term “repeat unit” or “monomer” means a group in a polymer that repeats or appears multiple times in a polymer.

Exemplary polymers include but are not limited to polyethylene, polyacrylamides, polymethacrylamides, polyacrylates, polymethacrylates, proteins, carbohydrates, polyvinylamine, and polyallylamine. Other polymers will be readily apparent to those of skill in the art given the benefit of the present disclosure.

The term “co-polymer” means a polymer with two or more repeat units where the repeat units or “comonomers” are chemically and structurally different from one another. Exemplary co-polymers include but are not limited to ethylene-vinylacetate, styrene-acrylonitrile, and styrene-isoprene-styrene. Other co-polymers will be readily apparent to those of skill in the art given the benefit of the present disclosure.

The term “pharmaceutically acceptable anion” means an anion that is suitable for pharmaceutical use. Pharmaceutically acceptable anions include but are not limited to chloride, bromide, iodide, carbonate, bicarbonate, sulfate, nitrate, phosphate, acetate, ascorbate, benzoate, citrate, dihydrogen citrate, hydrogen citrate, oxalate, succinate, tartrate, taurocholate, glycocholate, cholate, fumarate, lactate, malate, tosylate, valerate, mucate, diphosphate and maleate.

A “guanidino group” is represented by Formula (A):

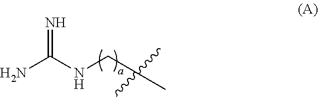

wherein a is an integer from 0 to 25,

A “guanidinium chloride group” is represented by Formula (B),

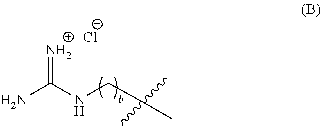

wherein b is an integer from 0 to 25,

A “guanidinobenzene group” is represented by Formula (C),

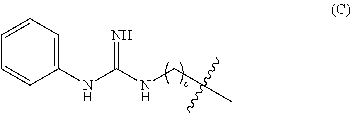

wherein c is an integer from 0 to 25,

A “dihydroxy group” is represented by Formula (D),

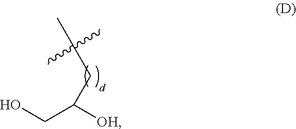

wherein d is an integer from 0 to 25, or

A “polyethylene glycol group” (PEG) is represented by Formula (E)

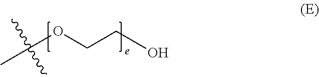

wherein e is an integer from 1 to 400.

The term “dicarbonyl” refers to an organic molecule containing two or more adjacent carbonyl groups. Carbonyl groups, represented by C═O, can be, for example, aldehydes, ketones, and other groups with an oxygen atom doubly bonded to a carbon atom. Examples include but are not limited to glyoxal, methylglyoxal, dimethyl glyoxal, and 3-deoxyglucosone.

The term “pharmaceutically acceptable end group” means an end group that is suitable for pharmaceutical use. Examples of pharmaceutically acceptable end groups include but are not limited to H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a guanidino group, a guanidinium chloride group, a guanidinobenzene group, a dihydroxy group, and a polyethylene glycol group.

### Related Art

Not applicable

### BRIEF SUMMARY OF THE INVENTION

One aspect of the invention relates to a pharmaceutical composition comprising a compound, wherein the compound comprises the structure of Formula I:

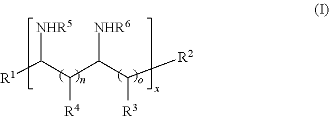

wherein:
n is 0, 1, or 2;
o is 0, 1, or 2;
x is an integer from 2 to 25,000;
R¹ and R² are each independently a pharmaceutically acceptable end group, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer, wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
   or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂, or
R⁵ and R⁶ are taken together with the nitrogens to which they are attached to form a 6 to 20 member ring.

In one aspect of the invention, it is directed to a pharmaceutical composition comprising a compound, wherein the compound comprises the structure of Formula I-A:

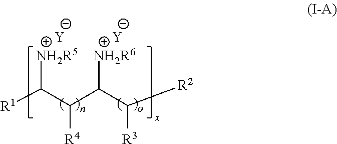

wherein:
n is 0, 1, or 2;
o is 0, 1, or 2;
x is an integer from 2 to 25,000;
Y⁻ is each independently a pharmaceutically acceptable anion;
R¹ and R² are each independently a pharmaceutically acceptable end group, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

Another aspect of the invention relates to a pharmaceutical composition comprising a compound, wherein the compound comprises the structure of Formula II:

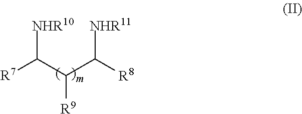

wherein:
m is 0, 1, or 2;
R⁷ and R⁸ are each independently a pharmaceutically acceptable end group or a polymer, —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or
R⁷ and R⁸ are taken together with the carbons to which the) are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer,
      wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide;
R⁹ is H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a polymer, or —R^{y}-polymer,
   wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide,
or if m is 0, R⁹ is absent; and
R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R¹⁰ and R¹¹ are taken together with the nitrogens to which they are attached to form a 6 to 20 member ring.

Yet another aspect of the invention relates to a pharmaceutical composition comprising a compound, wherein the compound comprises the structure of Formula II-A:

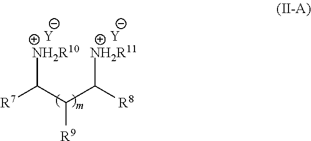

wherein:
m is 0, 1, or 2;
Y⁻ is each independently a pharmaceutically acceptable anion;
R⁷ and R⁸ are each independently a pharmaceutically acceptable end group or a polymer, —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or
R⁷ and R⁸ are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer,
      wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide; and
R⁹ is H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a polymer, or —R^{y}-polymer,
   wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide,
or if m is 0, R⁹ is absent; and
R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R¹⁰ and R¹¹ are taken together with the nitrogens to which they are attached to form a 6 to 20 member ring.

In another aspect, the invention relates to a method of binding AGE precursors in a mammal comprising administering to the patient a pharmaceutical composition comprising a compound according to Formula I. In yet another aspect, the invention relates to a method of binding AGE precursors in a mammal comprising administering to the patient a pharmaceutical composition comprising a compound according to Formula I-A. In another aspect, the invention relates to a method of binding AGE precursors in a mammal comprising administering to the patient a pharmaceutical composition comprising a compound according to Formula II. In yet another aspect, the invention relates to a method of binding AGE precursors in a mammal comprising administering to the patient a pharmaceutical composition comprising a compound according to Formula II-A.

In another aspect, the invention relates to a method of binding dietary dicarbonyls in a mammal comprising administering to the patient a pharmaceutical composition comprising a compound according to Formula I. In yet another aspect, the invention relates to a method of binding dietary dicarbonyls in a mammal comprising administering to the patient a pharmaceutical composition comprising a compound according to Formula I-A. In another aspect, the invention relates to a method of binding dietary dicarbonyls in a mammal comprising administering to the patient a pharmaceutical composition comprising a compound according to Formula II. In yet another aspect, the invention relates to a method of binding dietary dicarbonyls in a mammal comprising administering to the patient a pharmaceutical composition comprising a compound according to Formula II-A.

### BRIEF DESCRIPTION OF THE SEVERAL VIEWS OF THE DRAWING(S)

FIG. 1 illustrates the structures of several examples of dicarbonyl compounds in foods, or formed either by digestion and/or biological oxidation/peroxidation.

FIG. 2 shows a comparison of dicarbonyl sequestration (MGO binding properties) between 10% epichlorohydrin crosslinked high molecular weight poly(vinylamine) and sevelamer carbonate at pH 5.8, as described in Example 1-2.

FIG. 3 shows a comparison of dicarbonyl sequestration (MGO binding properties) between 10% epichlorohydrin crosslinked high molecular weight poly (vinylamine) and sevelamer carbonate at pH 3, as described in Example 1-2.

FIG. 4 illustrates the effect of 10% epichlorohydrin crosslinked high molecular weight poly(vinylamine) on the inhibition of the formation of plasma CML in Uremic Rats, as described in Example 2-1.

FIG. 5 illustrates the effect of sevelamer carbonate on the inhibition of the formation of plasma CML in Uremic Rats, as described in Example 2-2.

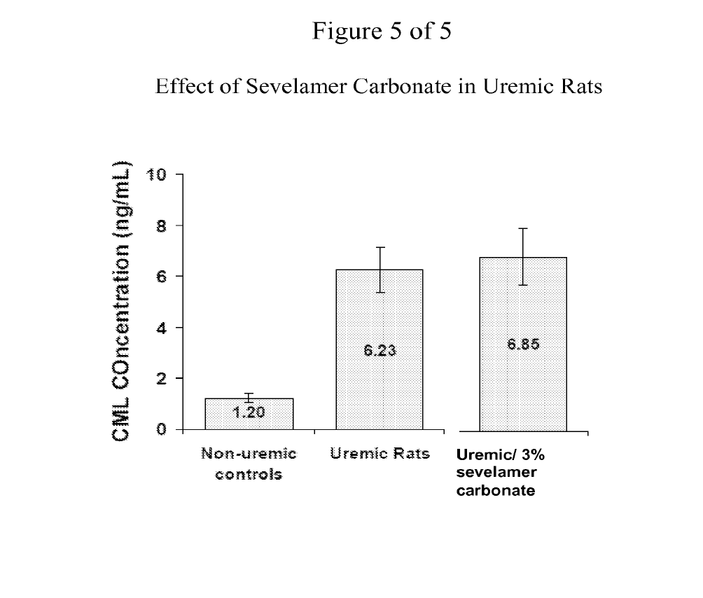

### DETAILED DESCRIPTION OF THE INVENTION

This invention relates to novel sequestrants of advanced glycation end products (AGE) precursors. The sequestrants of AGE precursors are of varying structures and comprise amine groups.

Advanced glycation end products are modified proteins and protein derivatives that are formed by the reaction of amino acid side chain functional groups, including but not limited to amine groups and guanidinium groups, with dicarbonyl compounds. Dicarbonyl compounds are present in foods, are formed during digestion, or are produced in the body through various biochemical processes. Dicarbonyls present in foods or formed in the gut during digestion can be absorbed into the body where they can react with proteins to form AGE. It is an object of the present invention to prevent that absorption by reacting the dicarbonyls present in the gastrointestinal tract to the materials of the present invention, causing them to be safely excreted in the feces before they can be absorbed.

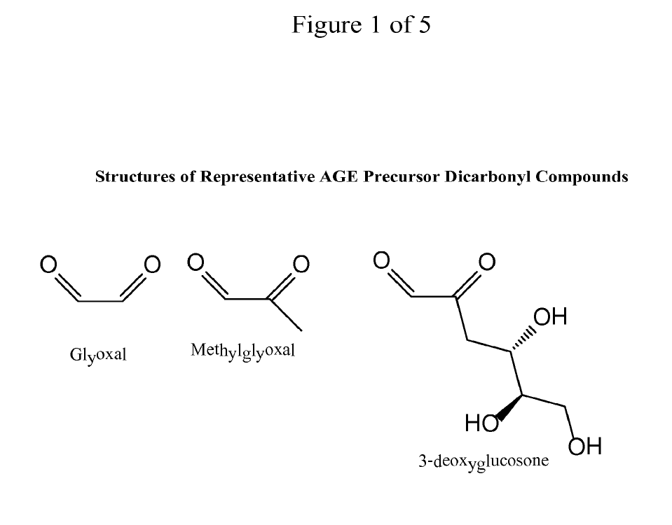

AGE are formed by the reaction of dicarbonyl compounds with amino acid side chains of proteins through the Maillard reaction. The pendant amino groups of lysine residues of a protein react with carbonyl compounds to form a Schiff base. The Schiff base, under physiological conditions, transforms through a process called Amadori rearrangements. Dicarbonyls also react with arginine and other amine- and guanidine-containing biomolecules through analogous processes. The resulting compounds, AGE, are toxic due to protein crosslinking, among other toxic mechanisms. Structures of several dicarbonyl compounds in foods, or formed either by digestion and/or biological oxidation/peroxidation are shown in FIG. 1.

Formation of AGE is accelerated by metabolic diseases such as chronic kidney disease (CKD). Formation and accumulation of AGE in the plasma and tissue lead to a number of disorders of cardiovascular and renal complications including atherosclerosis, and diabetic nephropathy. The present invention addresses a novel approach to suppress the formation of AGE by selectively removing the dietary dicarbonyls and endogenous dicarbonyls found in food or produced in the gut using appropriate sequestrants of AGE precursors. The sequestrants of AGE precursors bind the carbonyl compounds through chemoselective reactions. These high molecular weight sequestrants of AGE precursors (preferably polymers that are crosslinked or non-crosslinked) are biostable and systemically non-absorbed. As a result, the sequestered dicarbonyls are removed through fecal excretion. The sequestrants of AGE precursors of the present invention can be soluble high molecular weight polymers and crosslinked polymer hydrogels compositions containing amine groups. The invention discloses that these materials are useful as therapeutically significant agents to sequester dicarbonyl compounds in the GI tract for the treatment of a number of ailments such as diabetic nephropathy, chronic renal disease, atherosclerosis, stroke, cataracts, and Alzheimer's disease.

The sequestrants of AGE precursors contain amine groups separated by 2, 3 or 4 carbons. The amine groups may be primary, secondary or tertiary amines. The sequestrants of AGE precursors can be small molecules or polymers. If the sequestrants of AGE precursors are polymers, they may be a polymer or copolymer and the amine groups may be on the polymer backbone or pendant from the polymer backbone.

The sequestrants of AGE precursors of the present invention are of varying molecular weights.

The sequestrants of AGE precursors bind diet derived dicarbonyl compounds in the gastrointestinal tract. The sequestered dicarbonyls are removed through fecal excretion.

This invention relates to pharmaceutical compositions comprising sequestrants of AGE precursors. This invention also relates to methods of binding AGE precursor compounds and method of binding dietary dicarbonyls with sequestrants of AGE precursors. The sequestrants of AGE precursors and the pharmaceutical compositions comprising sequestrants of AGE precursors can be administered in multiple dosage forms.

One embodiment of the present invention relates to sequestrants of AGE precursors comprising a compound with the structure of Formula I:

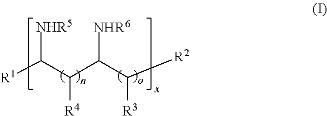

wherein:
n is 0, 1, or 2;
o is 0, 1, or 2;
x is an integer from 2 to 25,000;
R¹ and R² are each independently a pharmaceutically acceptable end group, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer, wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
   or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

Another embodiment of the present invention relates to sequestrants of AGE precursors wherein the compound comprises the structure of Formula I-A:

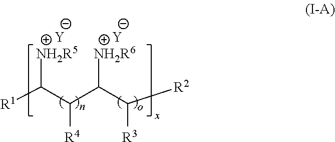

wherein:
n is 0, 1, or 2;
o is 0, 1, or 2;
x is an integer from 2 to 25,000;
Y⁻ is each independently a pharmaceutically acceptable anion;
R¹ and R² are each independently a pharmaceutically acceptable end group, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

Another embodiment of the present invention relates to sequestrants of AGE precursors wherein the compound comprises the structure of Formula II:

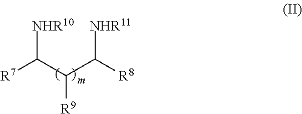

wherein:
m is 0, 1, or 2;
R⁷ and R⁸ are each independently a pharmaceutically acceptable end group or a polymer, —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or
R⁷ and R⁸ are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer,
      wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide:
R⁹ is H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a polymer, or —R^{y}-polymer,
   wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide,
or if m is 0, R⁹ is absent; and
R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R¹⁰ and R¹¹ are taken together with the nitrogens to which they are attached to form a 6 to 20 member ring.

Another embodiment of the present invention is directed to sequestrants of AGE precursors wherein the compound comprises the structure of Formula II-A:

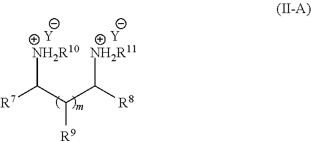

wherein:
m is 0, 1, or 2;
Y⁻ is each independently a pharmaceutically acceptable anion;
R⁷ and R⁸ are each independently a pharmaceutically acceptable end group or a polymer, —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or
R⁷ and R⁸ are taken together with the carbons to which they are attached to form a 3 to 10 member ring.
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer,
      wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₀)cycloalkyl-COOH, —(O)CH₃, —OH, or amide; and
R⁹ is H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a polymer, or —R^{y}-polymer,
   wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide,
or if m is 0, R⁹ is absent; and
R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R¹⁰ and R¹¹ are taken together with the nitrogens to which they are attached to form a 6 to 20 member ring.

In one embodiment this invention comprises to a method of binding AGE precursors in a mammal comprising administering a pharmaceutical composition comprising a sequestrant of AGE precursors, wherein the sequestrant of AGE precursors comprises a compound with the structure of Formula I, Formula I-A, Formula II or Formula II-A.

In another embodiment this invention comprises to a method of binding dietary dicarbonyls in a mammal comprising administering a pharmaceutical composition comprising a sequestrant of AGE precursors, wherein the sequestrant of AGE precursors comprises a compound with the structure of Formula I, Formula I-A, Formula II or Formula II-A.

In another embodiment of the invention, the sequestrants of AGE precursors are the active pharmaceutical ingredient in a pharmaceutical composition. In a preferred embodiment of the invention, the pharmaceutical composition comprises a compound, wherein the compound comprises the structure of Formula I:

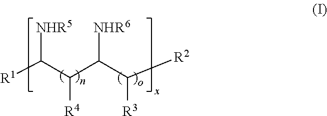

wherein:
n is 0, 1, or 2;
o is 0, 1, or 2;
x is an integer from 2 to 25,000;
R¹ and R² are each independently a pharmaceutically acceptable end group, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide:
R³ and R⁴ are each independently H, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
   or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

In another preferred embodiment of the invention, the pharmaceutical composition comprises a compound, wherein the compound comprises the structure of Formula I-A:

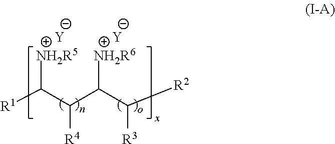

wherein:
n is 0, 1, or 2;
o is 0, 1, or 2;
x is an integer from 2 to 25,000;
Y⁻ is each independently a pharmaceutically acceptable anion;
R¹ and R² are each independently a pharmaceutically acceptable end group, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

In another embodiment of the invention the sequestrants of AGE precursors are the active pharmaceutical ingredient in a pharmaceutical composition. In a preferred embodiment of the invention, the pharmaceutical composition comprises a compound, wherein the compound comprises the structure of Formula II:

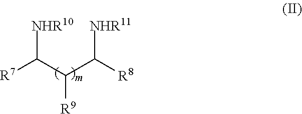

wherein:
m is 0, 1, or 2;
R⁷ and R⁸ are each independently a pharmaceutically acceptable end group or a polymer, —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or
R⁷ and R⁸ are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer,
      wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide:
R⁹ is H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a polymer, or —R^{y}-polymer,
   wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide,
or if m is 0, R⁹ is absent; and
R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R¹⁰ and R¹¹ are taken together with the nitrogens to which they are attached to form a 6 to 20 member ring.

In another embodiment of the invention, the sequestrants of AGE precursors are the active pharmaceutical ingredient in a pharmaceutical composition. In a preferred embodiment of the invention, the pharmaceutical composition comprises a compound, wherein the compound comprises the structure of Formula II-A:

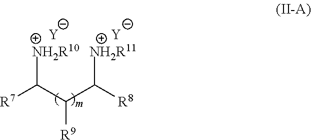

wherein:
m is 0, 1, or 2;
Y⁻ is each independently a pharmaceutically acceptable anion;
R⁷ and R⁸ are each independently a pharmaceutically acceptable end group or a polymer, —R^{x}-polymer,
   wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or
R⁷ and R⁸ are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer,
      look for periods wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide; and
R⁹ is H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a polymer, or —R^{y}-polymer,
   wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide,
or if m is 0, R⁹ is absent; and
R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R¹⁰ and R¹¹ are taken together with the nitrogens to which they are attached to form a 6 to 20 member ring.

In one embodiment of the invention, the sequestrants of AGE precursors are polymers. In some embodiments, the polymers may comprise a monomer comprising a compound having a repeat unit according to Formula I, Formula I-A, Formula II or Formula II-A. In other embodiments, the polymers may comprise a monomer comprising a compound having two or more repeat units, where the upper limit is not thought to be critical. Accordingly, the sequestrants of AGE precursors may comprise a monomer repeating two to over a million times, preferably two to 25,000. In one embodiment of the invention, the sequestrants of AGE precursors are copolymers. In some embodiments, the copolymers may comprise a monomer comprising a compound having at least one unit which is copolymerized with one or more other comonomers or oligomers or other polymerizable groups.

In preferred embodiments of the invention, the sequestrants of AGE precursors are a compound of Formula I or Formula I-A where n and o are both 0, and where n and o are both 1. In another preferred embodiment, the sequestrants of AGE precursors are a compound of Formula II or Formula II-A, where m is 0. In another preferred embodiment, the sequestrants of AGE precursors are polymers. In a preferred embodiment of the invention the sequestrant of AGE precursors of Formula I or Formula I-A are poly(vinylamine). In another preferred embodiment the sequestrants of AGE precursors of Formula I or Formula I-A are poly(methyleneamine). In another preferred embodiment, the sequestrants of AGE precursors of Formula II or Formula II-A are poly{2,3-diamino{[3-[(2-methyl-1-oxo-2-propen-1-yl)amino]propyl]amino}propaneamide-co-ethylenebismethacrylamide}. In yet another preferred embodiment, the sequestrants of AGE precursors of Formula II or Formula II-A are poly(3,4-diaminostyrene-co-divinyl benzene).

In a preferred embodiment of the invention, R¹ and R² are each independently and R⁷ and R⁸ are each independently a pharmaceutically acceptable end group. In another preferred embodiment of the invention, R⁷ and R⁸ are each independently a pharmaceutically acceptable end group, a polymer, —R^{x}-polymer, wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or R⁷ and R⁸ are taken together with the carbons to which they are attached to form a 3 to 10 member ring, wherein the 3 to 10 member ring is optionally attached to a polymer or substituted by one to four groups selected from (C₁-C₁₀)alkyl, (C₂-C₅)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer, wherein R^{y} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide.

In a more preferred embodiment, R¹ and R² are each independently and R⁷ and R⁸ are each independently H, a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a guanidino group, a guanidinium chloride, a guanidinobenzene group, a dihydroxy group, a polyethylene glycol group, a polymer, —R^{x}-polymer, wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, or, R⁷ and R⁸, are taken together with the carbons to which they are attached to form a 3 to 10 member ring, wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide or —R^{y}-polymer, wherein R is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, or amide.

In a preferred embodiment of the invention, R⁵ and R⁶ are each independently and R¹⁰ and R¹¹ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂. In a more preferred embodiment. R⁵ and R⁶ are each independently and R¹⁰ and R¹¹ are each independently H or (C₁-C₁₀)alkyl. In yet another preferred embodiment, R⁵ and R⁶ are each independently and R¹⁰ and R¹¹ are each independently H or —CH₃. In another preferred embodiment, R⁵ and R⁶ are each and R¹⁰ and R¹¹ are each H.

In a preferred embodiment of the invention, R⁵ and R⁶ are taken together with the nitrogens to which they are attached and R¹⁰ and R¹¹ are taken together with the nitrogens to which they are attached to form a 6 to 20 member ring. In a preferred embodiment, R⁵ and R⁶ are taken together with the nitrogens to which they are attached to form a 14 member ring. In another preferred embodiment, R¹⁰ and R¹¹ are taken together with the nitrogens to which they are attached to form a 14 member ring.

In a preferred embodiment of the invention, Y⁻ is a pharmaceutically acceptable anion. In another preferred embodiment, Y⁻ is independently selected from carbonate, bicarbonate or chloride. In another preferred embodiment, Y⁻ is independently selected from carbonate or bicarbonate. In another preferred embodiment, Y⁻ is chloride.

In preferred embodiment, the sequestrants of AGE precursors of Formula I, Formula I-A, Formula II or Formula II-A are crosslinked polymers. The sequestrants of AGE precursors of Formula I, Formula I-A, Formula II or Formula II-A are crosslinked with epichlorohydrin, represented by Formula F.

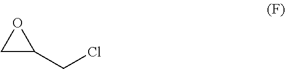

Non-limiting examples of suitable sequestrants of AGE precursors according to Formula I, Formula I-A, Formula II or Formula II-A are presented in Table 1. It is understood that any or all of the amines of the structures presented in Table 1 may be in the free amine form or in a protonated form with a pharmaceutically acceptable anion. Preferred pharmaceutically acceptable anions include but are not limited to chloride, bromide, iodide, carbonate, bicarbonate, sulfate, nitrate, phosphate, acetate, ascorbate, benzoate, citrate, dihydrogen citrate, hydrogen citrate, oxalate, succinate, tartrate, taurocholate, glycocholate, cholate, fumarate, lactate, malate, tosylate, valerate, mucate, diphosphate and maleate. Most preferred pharmaceutically acceptable anions include chloride, carbonate, and bicarbonate.

TABLE 1
Amine Based Sequestrants of AGE precursors

| Structure | Description |
|---|---|
| 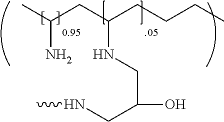 | 5% epichlorohydrin crosslinked low molecular weight poly(vinylamine) |
| 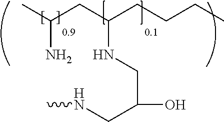 | 10% epichlorohydrin crosslinked low molecular weight poly(vinylamine) |
| 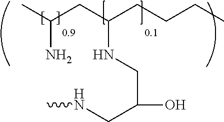 | 10% epichlorohydrin crosslinked low molecular weight poly(vinylamine) |
| 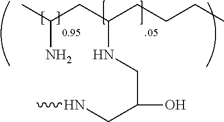 | 5% epichlorohydrin crosslinked low molecular weight poly(vinylamine) |
| 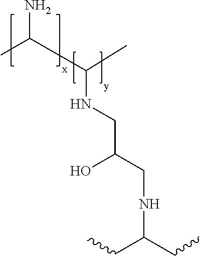 | 10% epichlorohydrin crosslinked poly(methyleneamine) |
| 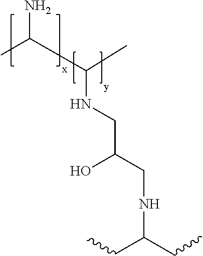 | 5% epichlorohydrin crosslinked poly(methyleneamine) |
| 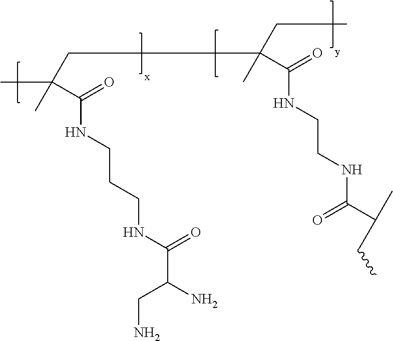 | Poly{2,3-diamino{[3-[(2- methyl-1-oxo-2-propen-1- yl)amino]propyl] amino}propaneamide- co- ethylenebismethacrylamide} (98:2) |
| 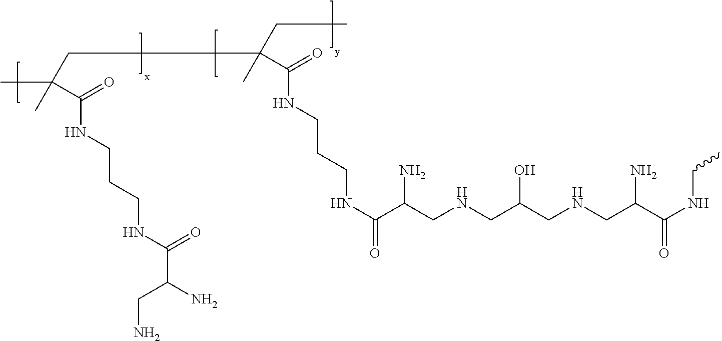 | 4% epichlorohydrin crosslinked poly{2,3- diamino{[3-[(2-methyl-1- oxo-2-propen-1- yl)amino]propyl] amino}propaneamide} |
| 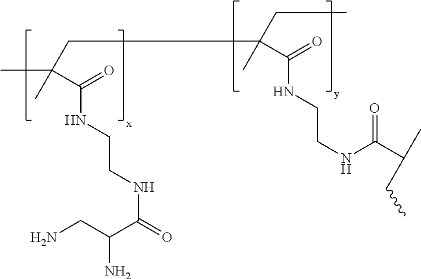 | Poly{2,3-diamino{[3-[(2- methyl-1-oxo-2-propen-1- yl)amino]propyl] amino}propaneamide-co- ethylenebismethacrylamide} (95:5) |
| 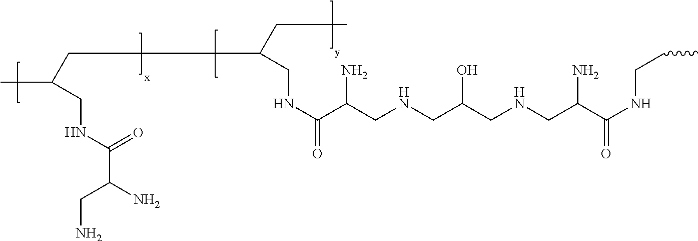 | 4% epichlorohydrin crosslinked poly(N-allyl-2,3- diamino-propane) |
| 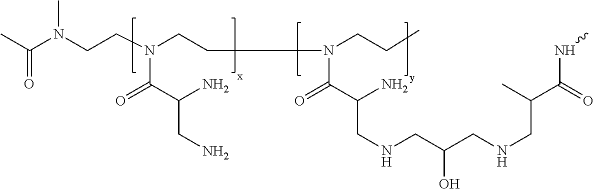 | Epichlorohydrin cross-linked poly[2-(2,3- diaminoethyl)oxazoline |
| 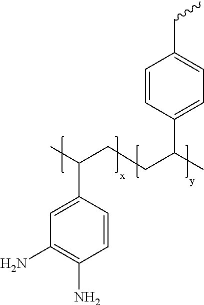 | Poly(3,4-diaminostyrene-co- divinyl benzene) (95:5) |
| 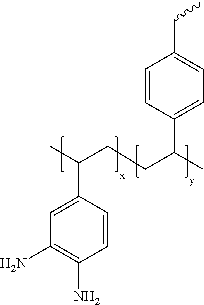 | Poly(3,4-diaminostyrene-co- divinyl benzene) (98:2) |
| 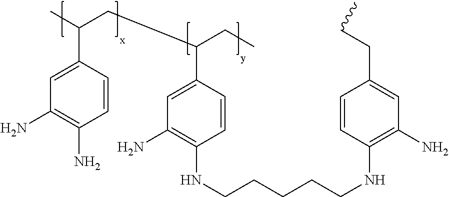 | Glutaraldehyde crosslinked poly(3,4-diaminostyrene) |
| 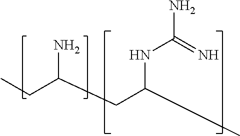 | Epichlorohydrin crosslinked Poly[(vinylamine)-co- (vinylguanidine)] |
| 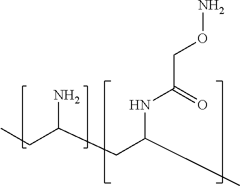 | Epichlorohydrin crosslinked Poly[(2-(aminooxy)-N- (vinyl)-acetamide)-co- (vinylamine)] |
| 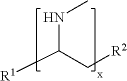 | Poly(N-methylvinylamine) |
| 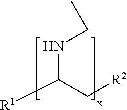 | Poly(N-ethylvinylamine) |
| 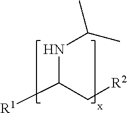 | Poly(N-isopropylvinylamine) |
| 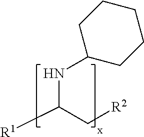 | Poly(N- cyclohexylvinylamine) |
| 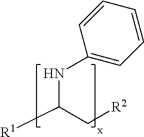 | Poly(N-vinylaniline) |
| 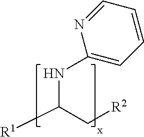 | Poly(N-vinylpyridin-2- amine) |
| 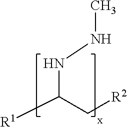 | Poly(N-methylallylamine) |
| 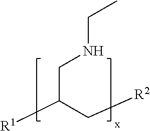 | Poly(N-ethylallylamine) |
| 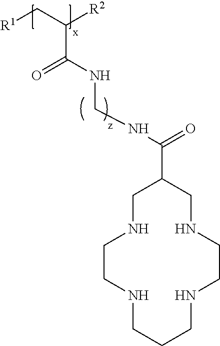 | Poly(N-(2-acrylamido[y]yl)- 1,4,8,11- tetraazacyclotetradecane-6- carboxamide), |
| 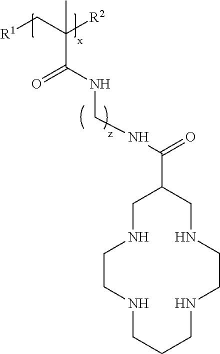 | Poly(N-(2- methacrylamido[y]yl)- 1,4,8,11- tetraazacyclotetradecane-6- carboxamide) |
| 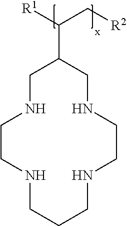 | Poly(6-vinyl-1,4,8,11- tetraazacyclotetradecane) |
| 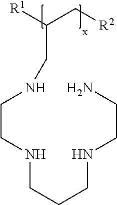 | Poly{N1-(2- (allylamino)ethyl)-N3-(2- aminoethyl)propane-1,3- diamine} |
| 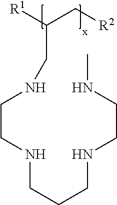 | Poly{N1-(2- (allylamino)ethyl)-N3-(2- (methylamino)ethyl)propane- 1,3-diamine} |

In an embodiment of the invention, the sequestrants of AGE precursors are administered in an effective amount to achieve the desired therapeutic effect. The skilled artisan will be able to determine the effective amount of the sequestrants of AGE precursors depending on the individual and the condition being treated.

In one embodiment of the invention, the sequestrants of AGE precursors and pharmaceutical compositions comprising sequestrants of AGE precursors can be used to bind AGE precursor compounds and dietary dicarbonyl compounds. The sequestrants of AGE precursors of the present invention may be administered alone or in a pharmaceutical composition comprising a sequestrant of AGE precursors or multiple sequestrants of AGE precursors. Suitable pharmaceutical compositions may comprise a sequestrant of AGE precursors and one or more pharmaceutically acceptable excipients. The form in which the sequestrant of AGE precursors are administered, for example, powder, tablet, capsule, solution, or emulsion, depends in part on the route by which it is administered. The sequestrants of AGE precursors can be administered, for example, orally. Suitable excipients include, but are not limited to, are inorganic or organic materials such as gelatin, albumin, lactose, starch, stabilizers, melting agents, emulsifying agents, salts and buffers. Suitable pharmaceutically acceptable excipients for topical formulations such as ointments, creams and gels include, but are not limited to, commercially available inert gels or liquids supplemented with albumin, methyl cellulose, or a collagen matrix.

The sequestrants of AGE precursors and pharmaceutical compositions comprising sequestrants of AGE precursors can be administered alone or in combination with one or more additional drugs. Additional drugs administered in combination with the sequestrants of AGE precursors and pharmaceutical compositions comprising sequestrants of AGE precursors of the present invention include therapies for the treatment of diabetic nephropathy, chronic kidney disease, atherosclerosis, stroke, cataract, and Alzheimer's disease. The additional drugs may be administered concomitantly with the sequestrants of AGE precursors or pharmaceutical compositions comprising sequestrants of AGE precursors. The additional drugs may also be administered in series with the sequestrants of AGE precursors or pharmaceutical compositions comprising sequestrants of AGE precursors. The pharmaceutical composition comprising sequestrants of AGE precursors may also further comprise a drug used prophylactically and/or therapeutically for the treatment or prevention of diabetic nephropathy, chronic kidney disease, atherosclerosis, stroke, cataract, and Alzheimer's disease.

In one embodiment of the invention, the number of repeat units and the molecular weight are controlled by the synthesis of the sequestrants of AGE precursors. Methods of preparing preferred sequestrants of AGE precursors of the invention and controlling for the number of repeat units and molecular weights are described in Example 3.

### EXAMPLES

### Example 1: In Vitro Studies

### Example 1-1: Dicarbonyl Sequestration

A solution of methylglyoxal (MGO) at a concentration of 50 mg/mL in an aqueous buffer containing 100 mM each of sodium chloride and 2-(N-morpholino)ethanesulfonic acid (MES) with a pH of 5.8 was prepared. To achieve a pH of 3.0, the solution was titrated with IM hydrochloric acid.

50 mg of polymeric sequestrant of AGE was added to 50 mL of each MGO solution (pH 5.8 and pH 3.0). The reaction mixture was stirred and an aliquot was taken at appropriate times, ranging from 5 minutes to 24 hours, at minimum an aliquot was taken at 60 minutes. The amount of MGO present in the test solution, after the timed exposure to the polymer and subsequent filtration to remove the polymer, was determined by gas chromatography after derivatizing the MGO with o-phenylenediamine. The amount of MGO bound to the sequestrants of AGE precursors was determined by subtracting the residual MGO present in the binding solution from the starting concentration of MGO. The MGO binding properties (mg of MGO/g of sequestrants of AGE precursors) at a pH of 5.8 at 60 minutes are presented in Table 2 below.

TABLE 2
In vitro Methylglyoxal (MGO) Binding Properties of
Sequestrants of AGE Precursors at 60 minutes, pH 5.8

| Sequestrant of AGE | MGO Bound* |
|---|---|
| 5% epichlorohydrin crosslinked low | 47 |
| molecular weight poly(vinylamine) | |
| 10% epichlorohydrin crosslinked low | 48 |
| molecular weight poly(vinylamine) | |
| 10% epichlorohydrin crosslinked high | 48 |
| molecular weight poly(vinylamine) | |
| 5% epichlorohydrin crosslinked high | 47 |
| molecular weight poly(vinylamine) | |
| Epichlorohydrin crosslinked | 32 |
| Poly[(vinylamine)-co-(vinylguanidine)] | |
| Epichlorohydrin crosslinked Poly[(2- | 43 |
| (aminooxy)-N-(vinyl)-acetamide)-co- | |
| (vinylamine)] | |
| Poly(3,4-diaminostyrene-co-divinyl | 12 |
| benzene) (95:5) | |
| Poly(3,4-diaminostyrene-co-divinyl | 5 |
| benzene) (98:2) | |
| Poly{2,3-diamino{[3-[(2-methyl-1-oxo-2- | 43 |
| propen-1-yl)amino]propyl]amino}propaneamide- | |
| co- | |
| ethylenebismethacrylamide} (98:2) | |
| 5% epichlorohydrin crosslinked | 49 |
| poly(methyleneamine) | |
| 10% epichlorohydrin crosslinked | 33 |
| poly(methyleneamine) | |

*mg MGO/g sequestrant of AGE precursors

### Example 1-2: Comparative Dicarbonyl Sequestration: Sequestrants of AGE Precursors and Sevelamer Carbonate

Conditions were established to mimic the environment of the stomach and small intestine to conduct comparative in vitro binding studies with 10% epichlorohydrin crosslinked high molecular weight poly(vinylamine) and sevelamer carbonate. A solution of methylglyoxal (MGO) at a concentration of 50 mg/mL in an aqueous buffer containing 100 mM each of sodium chloride and 2-(N-morpholino)ethanesulfonic acid (MES) with a pH of 5.8 was prepared. To achieve a pH of 3.0, the solution was titrated with IM hydrochloric acid. These solutions were intended to mimic the amount of AGE precursors and dietary dicarbonyls present in the stomach and small intestines after a meal.

50 mg of test compound (10% epichlorohydrin crosslinked high molecular weight poly(vinylamine) or sevelamer carbonate) was added to 50 mL of each MGO solution at each pH 5.8. The reaction mixture was stirred and an aliquot was taken at appropriate times, ranging from 5 minutes to 24 hours. The amount of MGO present in the test solution, after the timed exposure to the test compounds and subsequent filtration to remove the test compounds, was determined by gas chromatography after derivatizing the MGO with o-phenylenediamine. The amount of MGO bound to the test compound was determined by subtracting the residual MGO present in the binding solution from the starting concentration of MGO.

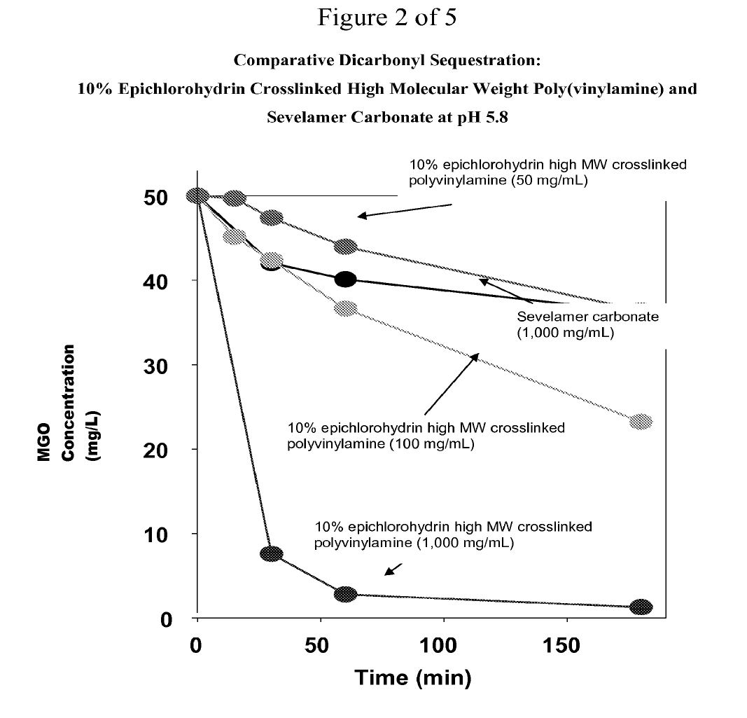

A comparison of the MGO binding properties (mg of MGO/g of the test compounds) at a pH of 5.8 is presented in FIG. 2 below. This comparative analysis demonstrated that 10% epichlorohydrin crosslinked high molecular weight poly(vinylamine) is 10-20 times more potent at binding MGO than sevelamer carbonate at pH 5.8.

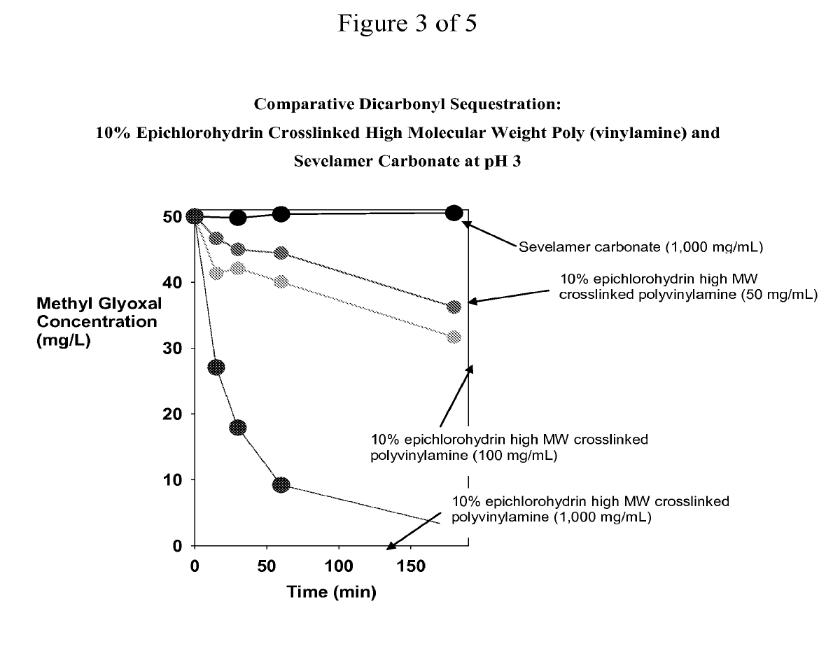

A comparison of the MGO binding properties (mg of MGO/g of the test compounds) at a pH of 3 is presented in FIG. 3 below. This comparative analysis demonstrated that 10% epichlorohydrin crosslinked high molecular weight poly(vinylamine) is 20 times more potent at binding MGO than sevelamer carbonate at pH 3.

### Example 2: In Vivo Studies

### Example 2-1: Effect of Sequestrants of AGE Precursors in Uremic Rats

Sprague Dawley rats were acclimated to the testing facility for 7 days. The rats were subsequently housed individually in metabolic cages and provided with a diet of rodent meal in food jars. After one week, 1% adenine was added to the diet. Adenine was then adjusted to 0.4% for two weeks to induce kidney impairment. Rats were given adenine free diet for another week. The following week, the rodents were provided diet mixed with 10% epichlorohydrin crosslinked high molecular weight poly(vinylamine).

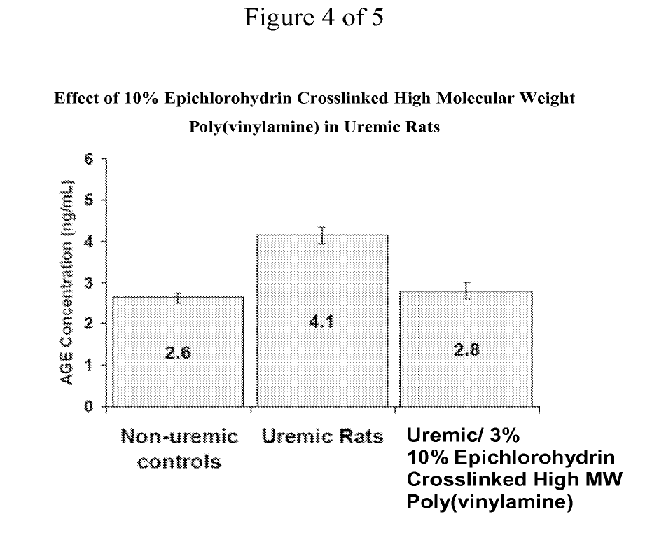

Blood samples were collected at the end of each week and analyzed for creatinine and carboxymethyl lysine (CML) using an ELISA assay. CML is produced by the reaction between MGO and lysine side chains of proteins. An increase CML value corresponds to AGE formation. The effect of 10% epichlorohydrin crosslinked high molecular weight poly(vinylamine) on the inhibition of the formation of plasma CML is displayed in FIG. 4. 10% epichlorohydrin crosslinked high molecular weight poly(vinylamine) effectively inhibited the formation of plasma CML.

### Example 2-2: Effect of Sevelamer Carbonate in Uremic Rats

Sprague Dawley rats were acclimated to the testing facility for 7 days. The rats were subsequently housed individually in metabolic cages and provided with a diet of rodent meal in food jars. After one week, 1% adenine was added to the diet. Adenine was then adjusted to 0.4% for two weeks to induce kidney impairment. The following week, the rodents were provided diet mixed with sevelamer carbonate.

Blood samples were collected at the end of each week and analyzed for creatinine and carboxymethyl lysine (CML) using an ELISA assay. CML is produced by the reaction between MGO and lysine side chains of proteins. An increase CML value corresponds to AGE formation. The effect of sevelamer carbonate on the inhibition of the formation of plasma CML is displayed in Sevelamer carbonate had no effect on the formation of plasma CML.

### Example 3: Synthesis of Polymeric Sequestrants of AGE

### Example 3-1: Synthesis of 5 Mol % Epichlorohydrin Crosslinked Low Molecular Weight Poly(vinylamine)

### Example 3-1-1: Synthesis of Low Molecular Weight Poly(vinylamine)

10.0 g of N-vinylformamide, 225 mg of AIBN, and 42 mL of isopropanol were mixed in a 100 mL 3-necked round bottom flask. After purging the reaction mixture for 30 minutes with a slow stream of nitrogen gas, the reaction mixture was stirred at 78° C. for 1 hour under nitrogen. After cooling to room temperature, 50 mL of deionized (DI) water was added to the reaction mixture. The resulting solution was poured into 500 mL of acetone. After stirring for 10 minutes, the solvent was removed. The residue was dissolved in 15 mL of DI water and precipitated into 500 mL of acetone. After filtration, the residue was dried under reduced pressure. The polymer was dissolved in 85 mL of DI water and, to this solution 14.6 g of sodium hydroxide solution (50% aqueous solution) was added. The reaction mixture was stirred at 75° C. for 24 hours. The solution was dialyzed using a 3,000 Dalton molecular weight cut off membrane to remove any low molecular weight impurities. The solution was lyophilized, yielding 2.75 g of the product as an off white solid.

### Example 3-1-2: Crosslinking of Low Molecular Weight Poly(vinylamine) with Epichlorohydrin

1.32 g of low molecular weight poly(vinylamine) (

Example 3-1-1) and 5.28 g of DI water were mixed in a 10 mL glass vial. The reaction mixture was allowed to stir until a homogeneous solution was formed. 125 microliter of epichlorohydrin was added to the solution. The reaction mixture was allowed to stir until a gel formed. The polymer gel was allowed to cure for 48 hours. After breaking into small pieces, the gel particles were suspended in 200 mL of DI water, stirred for 30 minutes, and filtered. The process was repeated twice more; the filtered gel was lyophilized yielding 1.2 g of the product as an off white solid.

### Example 3-2: Synthesis of 10 Mol % Epichlorohydrin Crosslinked Low Molecular Weight Poly(vinylamine)

1.32 g of low molecular weight poly(vinylamine) (Example 3-1-1) and 5.28 g of DI water were mixed in a 10 mL glass vial. The reaction mixture was allowed to stir until a homogeneous solution was formed. 250 microliter of epichlorohydrin was added to the solution. The reaction mixture was allowed to stir until a gel formed. The polymer gel was allowed to cure for 48 hours. After breaking into small pieces, the gel particles were suspended in 200 mL of DI water, stirred for 30 minutes, and filtered. The process was repeated twice more; the filtered gel was lyophilized yielding 1.24 g of the product as an off white solid.

### Example 3-3: Synthesis of 5 Mol % Epichlorohydrin Crosslinked High Molecular Weight Poly(vinylamine)

### Example 3-3-1: Synthesis of High Molecular Weight Poly(vinylamine)

10.0 g of N-vinylformamide, 130 mg of V50, and 42 mL of deionized water were mixed in a 250 mL 3-necked round bottom flask. After purging the reaction mixture for 30 minutes with a slow stream of nitrogen gas, the reaction mixture was stirred at 60° C. for 8 hours under nitrogen. After cooling to room temperature, the reaction mixture was poured into 500 mL of acetone. After stirring for 10 minutes, the solvent was removed. The residue was dissolved in 20 mL of DI water and precipitated into 500 mL of acetone. After filtration the residues was under reduced pressure. The resulting polymer was dissolved in 85 mL of DI water, and to this solution 14.6 g of sodium hydroxide solution (500/aqueous solution) was added. The reaction mixture was stirred at 75° C. for 24 hours. The solution was dialyzed using a 8,000 Dalton molecular weight cut off membrane to remove any low molecular weight impurities. The solution was lyophilized, yielding 5.45 g of the product as an off white solid.

### Example 3-3-2: Crosslinking of High Molecular Weight Poly(vinylamine) with Epichlorohydrin

2 g of the high molecular weight poly(vinylamine) (Example 3-3-1) and 18 mL of DI water were mixed in a 50 mL round bottom flask. The reaction mixture was allowed to stir until a homogeneous solution was formed. 182 microliter of epichlorohydrin was added to the solution. The reaction mixture was allowed to stir until a gel formed. The polymer gel was allowed to cure for 48 hours. After breaking into small pieces, the gel particles were suspended in 400 mL of DI water, stirred for 30 minutes, and filtered. The process was repeated twice more; the filtered gel was lyophilized yielding 1.48 g of the product as an off white solid.

### Example 3-4: Synthesis of 10 Mol % Epichlorohydrin Crosslinked High Molecular Weight Poly(vinylamine)

2 g of the high molecular weight poly(vinylamine) (Example 3-3-1) and 18 mL of DI water were mixed in a 50 mL round bottom flask. The reaction mixture was allowed to stir until a homogeneous solution was formed. 364 microliter of epichlorohydrin was added to the solution. The reaction mixture was allowed to stir until a gel formed. The polymer gel was allowed to cure for 48 hours. After breaking into small pieces, the gel particles were suspended in 400 mL of DI water, stirred for 30 minutes, and filtered. The process was repeated twice more; the filtered gel was lyophilized yielding 1.8 g of the product as an off white solid.

### Example 3-5: Synthesis of 5 Mol % Epichlorohydrin Crosslinked Poly(methyleneamine)

### Example 3-5-1: Synthesis of Poly(methyleneamine)

A slurry containing 50 g of 2,2-diethoxyethanamine and 80 g of ice was cooled to −40° C. 76 mL of 5M HCl was added to the slurry in a drop-wise manner. 50 g of potassium isocyanate dissolved in 100 mL of DI water was then added drop-wise to the reaction mixture. The resulting reaction mixture was stirred at reflux temperature for 2 hours. The reaction mixture was concentrated to a volume of 50 mL under reduced pressure. When cooled to room temperature, a white precipitate was formed. The precipitate was filtered and dried, yielding 35 g of the product (urea derivative). The urea derivative was treated with 1 L 0.05M H₂SO₄ in a 2.5 L flask. The resulting reaction mixture was stirred for 48 hours. The pH of the slurry was maintained at 7 by the addition of an appropriate amount of aqueous Ba(OH)₂ solution. At the end of the reaction, the reaction mixture was filtered. The filtrate was evaporated to dryness, yielding 16 g of 2-hydroxy imidazole as an off white solid.

The resulting 16 g of 2-hydroxy imidazole and 160 mL of acetic anhydride were mixed in a 500 mL 3-necked round bottom flask. The resulting solution was refluxed for 3 hours. The reaction mixture was filtered while hot; the filtrate was concentrated to 50 mL. The solution was placed in a freezer for 30 minutes and the resulting slurry was triturated by adding DI water. The slurry was poured into 500 mL of DI water, and the residue was filtered and washed with additional water. The residue was dissolved in methylene chloride and extracted twice with saturated NaHCO₃. After drying the organic phase over anhydrous MgSO₄, the methylene chloride was removed under reduced pressure. The crude product was purified by flash chromatography using an isocratic system of CH₂Cl₂, hexane, and acetone (5:5:1), yielding 14 g of N,N′-diacetyl-2-imidazolone.

1.62 g of N,N′-diacetyl-2-imidazolone and 1.18 mg of 1,1′-azobis (cyclohexanecarbonitrile) were mixed in a 10 mL pressure glass vial. The vial was sealed and, through several cycles of freeze-thaw by pump-nitrogen release, the reaction atmosphere made nitrogen. The polymerization was conducted at 130° C. for 3 hours.

After cooling to room temperature, the residue was dissolved in 8 mL of DMF and poured into 200 mL of methanol. The suspension was filtered and the supernatants were discarded. The precipitation cycle was repeated twice and the resulting residue was dried under reduced pressure, yielding 1.2 g of the polymer.

3.89 g of the resulting polymer, 11 mL of glycerol and 6.5 mL of aqueous 3.54M LiCl solution were mixed in a 25 mL reaction vial. 9.2 g of solid NaOH was added to the mixture. The resulting reaction mixture was refluxed at 150° C. for 24 hours. After cooling to room temperature, the slurry was diluted with water. The reaction mixture was cooled in an ice bath and acidified by slowly adding concentrated HCl. A precipitate was formed and separated by filtration. The resulting solid was subjected to the same process by maintaining the same stoichiometry of NaOH, LiCl and glycerol. After another 24 hours, the above procedure was repeated. The filtrate was dialyzed through a 10 kilodalton membrane filter with several rounds of water exchange. The dialyzed solution was lyophilized yielding 0.94 g of the polymer.

### Example 3-5-2: Crosslinking of Poly(methyleneamine) with Epichlorohydrin

200 mg of poly(methyleneamine) (Example 3-5-1) and 0.4 mL of DI water were mixed in a 5 mL glass reaction vial. The reaction mixture was stirred until a homogeneous solution was obtained. 3 drops of 50% sodium hydroxide solution were added to the solution, followed by 24 microliter of epichlorohydrin. The reaction mixture was stirred at room temperature for 14 hours and then stirred at 60° C. for 8 hours. The polymer gel was broken into small pieces and dialyzed through a 10 kilodalton dialysis membrane with multiple water exchanges. The dialyzed polymer gel was lyophilized yielding 170 mg of the polymer.

### Example 3-6: Synthesis of 2 Mol % Epichlorohydrin Crosslinked Poly(methyleneamine)

300 mg of poly(methyleneamine) (Example 3-5-1) and 0.5 mL of DI water were mixed in a 5 mL glass reaction vial. The reaction mixture was stirred until a clear solution was obtained. 4 drops of 50% sodium hydroxide solution were added to the solution, followed 18 microliter of epichlorohydrin. The reaction mixture was stirred at room temperature for 14 hours and at 60° C. for 8 hours. The polymer gel was broken into small pieces and dialyzed through a 10 k Da dialysis membrane with multiple water exchanges. The dialyzed polymer gel was lyophilized yielding 250 mg of the polymer.

### Example 3-7: Synthesis of poly(2,3-diamino {[3-[(2-methyl-1-oxo-2-propen-1-yl)amino]propyl]amino}propaneamide-co-ethylene bis-methacrylamide) (98:2)

### Example 3-7-1: Synthesis of 2,3-di(N-boc)aminopropanoic acid

5.23 g of 2,3-Diaminopropanoic acid hydrochloride was dispersed in 84 mL of dioxane:water (1:1) mixture. 26 mL of triethylamine was added to this suspension and resulted in a clear solution. 19.9 g of Boc anhydride and the reaction mixture was allowed to stir at room temperature for 16 hours. The volume of the reaction mixture was reduced to ˜20 mL under reduced pressure. 20 mL of 4M sodium hydroxide was added to this concentrated reaction mixture. Subsequently DI water was added in a drop-wise manner until the reaction mixture became homogenous. The aqueous phase was extracted with diethyl ether (2×100) mL). The aqueous phase was collected and 100 mL of ethyl acetate was added to it. The two phase system was stirred rapidly in a 500 mL round bottom flask. While stirring, 1.2M HCl was slowly added to the reaction mixture until the pH of the aqueous phase was ˜1.5. The phases were separated and the aqueous phase was extracted with ethyl acetate (2×100 mL). The combined organic phase was washed with 20 mL of brine and dried over anhydrous MgSO₄. After filtration, the reaction mixture was evaporated to dryness. The residue was treated with warm diethyl ether and filtered. The solvent was removed under reduced pressure. The ether treatment was repeated twice, yielding 10.8 g of the product as a white solid.

### Example 3-7-2: Synthesis of NHS ester of 2,3-Di(N-boc)aminopropanoic acid

2.0 g of 2,3-Di(N-boc)aminopropanoic acid (Example 3-7-1), 756 mg of N-hydroxysuccinimide, and 15 mL of dichloromethane were combined in a 250 mL round bottom flask. The resulting solution was cooled to 0° C. in an ice bath and 1.56 g (7.56 mmol) DCC was added. After stirring at 0° C. for 15 minutes, the reaction mixture was slowly allowed to warm to room temperature, and then stirred at room temperature for an additional 3 hours. The reaction mixture was again cooled to 0° C. and the precipitate that formed was filtered off. The residue was rinsed with cold dichloromethane and the filtrate was diluted with 10 mL of dichloromethane. The resulting solution was extracted with DI water (2×25 mL) followed by saturated NaHCO₃ solution (2×25 mL). The organic phase collected and then dried over Na₂SO₄. After filtration, the solvent was removed under reduced pressure. The residue was dissolved in 15 mL diethyl ether and kept at −20° C. for 16 hours. The solid precipitate that formed was filtered and rinsed with 5 mL of cold ether. After drying under reduced pressure, 2.44 g of the desired product was obtained as a white powder.

### Example 3-7-3: Synthesis of 2,3-diamino{[3-[(2-methyl-1-oxo-2-propen-1-yl)amino]propyl]amino}propaneamide

1.1 g of NHS ester of 2,3-di(N-boc)aminopropanoic acid (Example 3-7-2), 15 mL of acetonitrile and 1.4 mL of triethyl amine were combined in a 100 mL round bottom flask. 0.98 g of N-(3-aminopropyl)-methacrylamide hydrochloride was slowly added to the solution. The resulting reaction mixture was stirred at room temperature for 18 hours. After removing the solvent under reduced pressure, the residue was dissolved in minimum amount of dichloromethane and was purified by silica gel flash chromatography using a gradient of ethyl acetate/hexane. After combining appropriate fractions the solvent was removed under reduced pressure yielding 0.75 g of the desired product as an off white solid.

### Example 3-7-4: Synthesis of poly{2,3-diamino{[3-[(2-methyl-1-oxo-2-propen-1-yl)amino]propyl]amino}propaneamide-co-ethylene bis-methacrylamide} (98:2)

0.95 g of 2,3-Di(N-Boc)amino-[[3-[(2-methyl-1-oxo-2-propen-1-yl)amino]propyl]amino]-propaneamide (Example 3-7-3), 9.5 mg of V50. 8.7 mg of ethylene bismethacrylamide, and 1.8 mL of ethanol were added to a 10 mL glass vial. After bubbling with a slow stream of nitrogen for 30 minutes, the reaction mixture was kept at 70° C. for 30 hours. After cooling to room temperature, the reaction mixture was treated with 5 mL of 3 M methanolic HCl and was stirred at room temperature for 16 hours; the reaction mixture was then heated to 50° C. and stirred for an additional 3 hours. The methanol was removed and the polymer was dialyzed through a 10 kDa dialysis membrane for 48 hours. The resulting solution was lyophilized, yielding 0.54 g of the polymer.

### Example 3-8: Synthesis of 4 mol % epichlorohydrin crosslinked Poly{2,3-diamino{[3-[(2-methyl-1-oxo-2-propen-1-yl)amino]propyl] amino}propaneamide}

1.0 g of 2,3-Di(N-Boc)amino-[[3-[(2-methyl-1-oxo-2-propen-1-yl)amino]propyl]amino]-propaneamide (Example 3-7-3), 10.0 mg of V50, and 2 mL of ethanol were added to a 10 mL glass vial. After bubbling with a slow stream of nitrogen for 30 minutes, the reaction mixture was kept at 70° C. for 24 hours. After cooling to room temperature, the reaction mixture was treated with 5 mL of 3 M methanolic HCl and was stirred at room temperature for 16 hours; the reaction mixture was then heated to 50° C. and stirred for an additional 3 hours. The methanol was removed and the residue was dissolved in 5 mL of 1.2 M HCl. The resulting solution was dialyzed against DI water using a 10 k Da dialysis membrane for 48 hours. The dialyzed solution was lyophilized, yielding 0.6 g of the polymer.

0.1 g of the above polymer and 0.25 mL of DI water were added in a 5 mL reaction vial. After a clear solution formed, 22 mg of KOH was added, followed by 1 microliter of epichlorohydrin. The reaction mixture was allowed to stir at room temperature for 18 hours, during which time the polymer gel was formed. The gel was broken into small pieces and was dialyzed against DI water using a 10 kDa dialysis membrane for 48 hours. Lyophilization of the dialyzed gel offered 91 mg of the desired polymer as an off white solid.

### Example 3-9: Synthesis of poly{2,3-diamino{[3-[(2-methyl-1-oxo-2-propen-1-yl)amino]ethyl]amino}propaneamide-co-ethylene bis-methacrylamide} (95:5

### Example 3-9-1: Synthesis of N-(2-aminoethyl)methacrylamide

3.2 g of N-Boc-ethylenediamine, 3 mL of triethylamine, and 20 mL of acetonitrile were combined in a 250 mL three necked round bottom flask. The resulting solution was treated with 5.5 g of N-hydroxysuccinimide methacrylate dissolved in 20 mL of acetonitrile followed by another 3 mL of triethylamine. The reaction mixture was stirred at room temperature for 16 hours. The solvent was removed under reduced pressure and the residue was dissolved in tert-butylmethyl ether. The residual mass was dissolved in a two phase system of tert-butylmethyl ether and saturated Na₂CO₃. The aqueous phase was extracted with 20 mL of tert-butylmethyl ether. The combined organic phase was extracted with 10% citric acid followed by saturated NaHCO₃. The organic phase was collected and dried over MgSO₄. After filtration, the solvent was removed under reduced pressure. The residue was placed in a 50 mL round bottomed flask and treated with 10 mL of 4M HCl in 1,4-dioxane. The reaction mixture was stirred at room temperature for 18 hours. The suspension was centrifuged. After removing the supernatant, the residue was dissolved in 10 mL of DI water and lyophilized, yielding 1.2 g of the product as a white solid.

### Example 3-9-2: Synthesis of 2,3-diamino{[3-[(2-methyl-1-oxo-2-propen-1-yl)amino]ethyl] amino}propaneamide

1.55 g of 2,3-Di(N-boc) aminopropanoic acid (Example 3-7-1), 15 mL of tetrahydrofuran (THF) and 1.1 mL of triethylamine were combined in a 50 mL round bottom flask. The solution was stirred at 0° C. and 770 microliter of iso-butylchloroformate was added to the stirred solution in a drop-wise manner. The reaction mixture was allowed to warm to room temperature slowly and was stirred for one hour at room temperature. After adding 0.85 mL of triethylamine, a solution containing 1 g of N-[2-aminoethyl]methylacrylamide hydrochloride in 3 mL of N,N-dimethylformamide (DMF) was slowly added to the reaction mixture. The resulting reaction mixture was stirred at room temperature for 15 hours. The reaction mixture was filtered and the residue was washed with cold DI water and dried under reduced pressure. The dried solid was dissolved in 20 mL methanol; to this solution 6 mL of concentrated HCl was added in a drop-wise manner. After stirring the reaction mixture for 48 hours at room temperature, the solvent was removed under reduced pressure. The residue was dissolved in 15 mL of DI water and lyophilized, yielding 1.1 g of the monomer as a white solid.

### Example 3-9-3: Synthesis of poly{2,3-diamino{[3-[(2-methyl-1-oxo-2-propen-1-yl)amino]ethyl]amino}propaneamide-co-ethylene bis-methacrylamide} (95:5)

0.7 g of 2,3-diamino-[[2-[(2-methyl-1-oxo-2-propen-1-yl)amino]ethyl]amino]-propaneamide (Example 3-9-1), 7.0 mg of V50, 17.0 mg of ethylene bis-methacrylamide, and 1.3 mL of DI water were combined in a 10 mL glass vial. After bubbling with a slow stream of nitrogen for 20 minutes, the reaction mixture was stirred at 70° C. for 18 hours. The polymer gel that formed was treated with 20 mL of ethanol and stirred for 24 hours. The resulting gel was broken into small pieces and the suspension was centrifuged. The supernatant was decanted and the residue was washed with ethanol (2×20 mL). After removing ethanol, the residue was treated with 15 mL of 1.2M HCl and dialyzed against water using 12 kDa dialysis membrane for 48 hours. The dialyzed slurry was lyophilized, yielding 0.4 g of the polymer as an off white solid.

### Example 3-10: Synthesis of epichlorohydrin Crosslinked Poly(N-allyl-2,3-diaminopropaneamide)

### Example 3-10-1: Synthesis of Poly(N-allyl-2,3-diaminopropaneamide)

0.5 mL N-methylmorpholine (NMP) followed by 2 mL of methanol was added to a solution of 95 mg of poly(allylamine) dissolved in 5 mL of methanol. While stirring, 2.0 g of NHS ester of 2,3-di(N-boc)aminopropanoic acid (Example 3-7-1)) was added to the solution. The reaction mixture was stirred at room temperature until a clear solution formed. Subsequently it was stirred at 50° C. for six days. After cooling to room temperature, 3 mL of concentrated HCl was added, followed by 2 mL of water. The resulting reaction mixture was stirred at 50° C. for 15 hours. After cooling to room temperature, the reaction mixture was dialyzed through a 5 kDa dialysis membrane for 48 hours. The dialyzed polymer solution was lyophilized, yielding 0.16 g of the polymer.

### Example 3-10-2: Epichlorohydrin Crosslinking of poly(N-allyl-2,3-diaminopropaneamide)

153 mg of poly(2,3-diamino-[allylamino]-propaneamide) (Example 3-10-1) and 0.4 mL of DI water were combined in a 5 mL glass vial. When a clear solution formed, 150 mg sodium carbonate was added. While stirring, 2 μL of epichlorohydrin was added to the polymer solution. The reaction mixture was stirred at room temperature for 2 hours, followed by stirring at 60° C. for 15 hours. The polymer gel that formed was dialyzed against DI water using 10 kDa dialysis membrane for 48 hours. The dialyzed polymer was lyophilized, yielding 112 mg of the product.

### Example 3-11: Synthesis of Poly[2-(2,3-diaminoethyl)oxazoline)

### Example 3-11-1: Synthesis of 2-[2,3-di(N-boc)aminoethyl]oxazoline

1.1 g of 2,3-di(N-boc)aminopropanoic acid (Example 3-7-1), 0.88 g of 2-bromoethyl amine hydrobromide, 0.5 mL of NMM, and 32 mL of methanol were added to a 250 mL round bottom flask. 1.2 g of 4-(4,6-dimethoxy-1,3,5-triazin-2-yl)-4-methylmorpholinium chloride was added to the solution. The resulting reaction mixture was stirred at room temperature for one hour. After adding 0.85 g of KOH dissolved in 15 mL of methanol, the reaction mixture was stirred at reflux temperature for 3.5 hours. The solvent was removed and the residue was dissolved in 100 mL of tert-butylmethyl ether. The ether solution was extracted with DI water (2×100 mL) and the organic phase was dried over MgSO₄. After removing the solvent, the residue was purified by silica gel flash chromatography using a gradient of ethyl acetate and hexane as the eluting solvent system. Removal of the solvent yielded 0.8 g of the desired product.

### Example 3-11-2: Polymerization of 2-[2,3-di(N-boc)aminoethyl]oxazoline

0.58 g of 2-[2,3-di(N-boc)aminoethyl]oxazoline (Example 3-11-1) and 0.46 mL of anhydrous acetonitrile were added to an oven-dried 5 mL glass vial. 5.5 mg of 4,5-dihydro-2,3-dimethyloxazoliumtrifluoromethanesulfonate as a 4.3% solution in acetonitrile was added to the solution. The resulting reaction mixture was stirred at 80° C. for 5 days. After cooling to room temperature, 22 microliter of piperidine was added and the reaction mixture was stirred at room temperature for 48 hours. The reaction mixture was subsequently evaporated to dryness and the residue was dissolved in 3 mL of dichloromethane. 3 mL of trifluoroacetic acid was added to the resulting solution and then stirred at room temperature for 16 hours. After removing the solvent, the residue was treated with 15 mL of 1.2M HCl. After a clear solution was formed, 1 mL of concentrated HCl was added and the resulting reaction mixture was stirred at 50° C. for 18 hours. After cooling to room temperature, the solution was filtered through a 0.2 μm filter and the filtrate was lyophilized, yielding 0.4 g of poly[2-(2,3-diaminoethyl)-oxazoline).

### Example 3-12: Synthesis of poly(3,4-diaminostyrene-co-divinyl benzene) (98:2)

### Example 3-12-1: Synthesis of 4-vinyl-2-nitroaniline

5.76 g of 2,4,6,8-tetramethyltetravinylcyclotetrasiloxane, 0.25 g of palladium dibromide and 0.55 g of (2-biphenyl)di-tert-butylphosphine were added to a 250 mL, 3-neck round bottom flask. While maintaining a nitrogen atmosphere, 40 mL of IM tetrabutylammonium fluoride in THF was added to the flask, followed by 4.0 g of 4-bromo-2-nitroaniline dissolved in 10 mL of THF. The reaction was stirred at 50° C. for 16 hours under nitrogen atmosphere. After cooling to room temperature, 60 mL of diethyl ether was added and the reaction mixture was stirred at room temperature for 30 minutes. The reaction mixture was extracted with DI water (2×100 mL). The organic phase was allowed to pass through a plug of silica. The filtrate was evaporated to dryness and the residue was subjected to silica gel flash chromatography using a gradient of dichloromethane and hexane as the eluting solvent. The desired fractions were collected and evaporated to dryness, yielding 2.19 g of 4-vinyl-2-nitroaniline.

### Example 3-12-2: Synthesis of 3,4-diaminostyrene

3.45 g of 4-vinyl-2-nitroaniline (Example 3-12-1), 20.2 g of sodium sulfide nonahydrate, 30 mL of ethanol, and 30 mL of DI water were combined in a 250 mL round bottom flask. The reaction mixture was refluxed for 18 hours. After removing ethanol under reduced pressure, the aqueous phase was extracted with ethyl acetate (3×100 mL). The combined organic phase was washed with brine followed by drying over anhydrous MgSO₄. After filtration, 0.5 g of activated charcoal was added to the solution and the slurry was heated to reflux for 30 minutes. The slurry was filtered through a plug of celite. The filtrate was evaporated to dryness and the residue was purified by flash chromatography by using amine modified silica gel as the stationary phase and a gradient of ethyl acetate/hexane as the mobile phase. The desired fractions were combined and evaporated to dryness. The residue was recrystallized from a mixture of ethyl acetate/hexane, yielding 1.8 g of the product.

### Example 3-12-3: Synthesis of t-Boc Protected 3,4-diaminostyrene

1.75 g of 3,4-diaminostyrene (Example 3-12-2) and 20 mL of acetone were combined in a 100 mL 3-neck round bottom flask. To this solution, 8.54 g of t-Boc anhydride was added, followed by the addition of 20 mL of 1M sodium hydroxide. The reaction mixture was allowed to stir at room temperature for 84 hours. After filtration, the acetone was removed under reduced pressure. The residue was treated with 25 mL of ethyl acetate and 25 mL of DI water. After shaking, the phases were separated. The aqueous phase was extracted with 25 mL of ethyl acetate. The combined organic phase was washed with brine and dried over anhydrous MgSO₄. After filtration, the solvent was evaporated. The residue was treated with 50 mL of hexane, stirred for 30 minutes and evaporated to dryness. The residue was subsequently dissolved in 125 mL refluxing hexane. The solution was filtered and the filtrate was kept at −20° C. for 18 hours. The solid that formed was filtered and washed with 5 mL of cold hexane. The residue was dried under reduced pressure yielding 3.55 g of the desired product.

### Example 3-12-4: Synthesis of divinyl benzene Crosslinked 3,4-diaminostyrene Polymer

All manipulations were carried out by keeping exposure to light at the minimum. 1.0 g of t-Boc protected 3,4-diaminostyrene (Example 3-12-3), 8 mg of 1,4-divinylbenzene, 3 mL of toluene, and 10 mg of AIBN were combined in a 10 mL glass vial. After flushing the solution with a slow stream of nitrogen for 15 minutes, the reaction mixture was kept at 80° C. for 18 hours, yielding a swollen gel. The gel was treated with 6 mL of methanol followed by 6 mL of 3 M methanolic HCl. The resulting reaction mixture was stirred at room temperature for 18 hours, and then stirred at 45° C. for 3 hours. After removing the solvent, the residue was treated with 10 mL of 1.2 M HCl and dialyzed against DI water using a 12 kDa dialysis membrane for 48 hours. The gel was subsequently lyophilized, yielding 0.4 g of the desired product.

### Example 3-13: Synthesis of Glutaraldehyde Crosslinked poly(3,4-diaminostyrene)

### Example 3-13-1: Synthesis of Soluble poly(3,4-diaminostyrene)

All manipulations were performed with minimum exposure to light. 1.0 g of t-Boc protected 3,4-diaminostyrene (Example 3-12-3), 3 mL of toluene and 10 mg of AIBN were combined in a 10 mL glass vial. The resulting solution was bubbled with a slow stream of nitrogen for 15 minutes. The reaction vial was sealed and heated at 80° C. for 18 hours. After removal of toluene, the residue was dissolved in 6 mL methanol, followed by the addition of 6 mL of 3 M methanolic HCl. The reaction mixture was stirred at room temperature for 18 hours, and then stirred at 45° C. for 3 hours. The solvent was removed and the residue was dissolved in 10 mL of 1.2 M HCl. The resulting solution was dialyzed against DI water using a 12 kDa dialysis membrane for 48 hours. The dialyzed solution was lyophilized. yielding 0.65 g of the polymer.

### Example 3-13-2: Synthesis of 5 mol % Glutaraldehyde Crosslinked poly(3,4-diaminostyrene)

0.63 g of poly-3,4-diaminostyrene (Example 3-13-1) dissolved in 1.5 mL of DI water was added to 30.4 mg of 50 wt. % aqueous glutaraldehyde solution. While stirring, 0.2 g of sodium cyanoborohydride was added to this solution. The resulting reaction mixture was stirred at 45° C. for 48 hours. 5 mL of 1.2M HCl was added to the reaction mixture and stirred for an additional 24 hours. The reaction mixture was then dialyzed against DI water through a 10 kDa dialysis membrane for 48 hours. The dialyzed gel was lyophilized, yielding 0.3 g of the polymer gel.

### Example 3-14: Synthesis of ethylene bis-methacrylamide Crosslinked poly(arginine methacrylamide) (95:5)

### Example 3-14-1: Synthesis of arginine methacrylamide Monomer

1.6 g of potassium hydrogencarbonate, 12 mL of DI water and 1 g of arginine were combined in a 100 mL round bottom flask. 5 mL of acetone was added to this solution. The reaction mixture was cooled to 0° C. To this stirred cold solution, 0.6 g of methacryloyl chloride dissolved in 3 mL of dioxane was added in a drop-wise manner over a period of 10 minutes. The reaction mixture was stirred at 0° C. for 1 hour, and then stirred for 2 hours at room temperature. The pH of the reaction mixture was brought to 1.0 with concentrated HCl. After adding 20 mL of saturated sodium chloride solution, the reaction mixture was washed with ethyl acetate (3×25 mL). The ethyl acetate layer was subsequently discarded. The aqueous layer was extracted with a 1:1 (v/v) mixture of ethyl acetate:isopropanol (3×50 mL). The combined organic phase was concentrated under reduced pressure. Additional isopropanol was added and the solution was concentrated again. The deposited sodium chloride was removed by filtration, and the filtrate was evaporated to dryness, yielding 0.8 g of desired product as viscous colorless oil.

### Example 3-14-2: Synthesis of poly(arginine methacrylamide-co-ethylene bis-methacrylamide)

0.8 g of arginine methacrylamide hydrogen chloride (Example 3-14-1), 26 mg of ethylene bis-methacrylamide, and 2.2 mL of DI water were combined in a 10 mL glass vial. To this solution 7.3 mg of V50 was added. After flushing with a slow stream of nitrogen for 30 minutes, the vial was sealed and stirred at 65° C. for 16 hours. The polymer gel that formed was dispersed in 20 mL of 1 M HCl and centrifuged. The supernatant was removed and the process was repeated four more times. The residue was lyophilized, yielding 0.4 g of the polymer gel.

### Example 3-15: Synthesis of ethylene bis-methacrylamide Crosslinked poly(agmatine methacrylamide)

### Example 3-15-1: Synthesis of agmatine methacrylamide Monomer

4.14 g of potassium carbonate, 10 mL of DI water, and 2.28 g of agmatine sulfate and 5 mL of acetone were combined in a 100 mL round bottom flask. 2.1 g methacryloyl chloride dissolved in 2 mL of dioxane was added in a drop-wise over a period of 10 minutes while stirring at 0° C. The reaction mixture was stirred for additional 2 hours. Subsequently the reaction mixture was extracted with ethyl acetate (3×20 mL). The combined organic layer was dried over anhydrous MgSO₄. It was filtered and the filtrate was concentrated under reduced pressure. The residue was redissolved in 10 mL of THF. To this solution 1 mL of 4M HCl in dioxane was added. The precipitate that formed was isolated and dried under reduced pressure, yielding 1.2 g of the desired product.

### Example 3-15-2: Synthesis of poly(agmatine methacrylamide-co-ethylene bis-methacrylamide) (90:10)

0.92 g of agmatine methacrylamide HCl (Example 3-15-1), 77.1 mg of ethylene bis-methacrylamide, and 3 mL of DI water were combined in a 10 mL glass vial. After a clear solution was obtained, 9.22 mg of V50 was added. The resulting reaction mixture was bubbled with a slow stream of nitrogen for 30 minutes. The vial was sealed and stirred at 65° C. for 18 hours. The gel that formed was treated with 20 mL of 1 M HCl. After breaking the gel into small pieces, the resulting suspension was centrifuged. The supernatant was removed and the residue was collected. The 1 M HCl treatment and centrifugation process was repeated three times. The filtered residue was lyophilized, yielding 0.5 g of the desired polymer.

### Example 3-15-3: Synthesis of poly(agmatine methacrylamide-co-ethylene bis-methacrylamide) (95:5)

0.92 g of agmatine methacrylamide HCl (Example 3-15-1), 38.4 mg of ethylene bis-methacrylamide, and 3 mL of DI water were combined in a 10 mL glass vial. After a clear solution was obtained, 9.22 mg of V50 was added to this solution. The resulting reaction mixture was bubbled with a slow stream of nitrogen for 30 minutes. The vial was sealed and stirred at 65° C. for 18 hours. The gel that formed was treated with 20 mL of 1 M HCl. After breaking the gel into small pieces, the resulting suspension was centrifuged. The supernatant was removed and the residue was collected. The 1 M HCl treatment and centrifugation process was repeated three times. The filtered residue was lyophilized, yielding 0.45 g of the desired polymer.

### Example 3-16: Synthesis of Glyoxal Crosslinked poly(O-vinyl hydroxylamine-co-vinyl alcohol)

### Example 3-16-1: Synthesis of poly(O-vinyl hydroxylamine-co-vinyl alcohol)

2.0 g of poly(vinyl alcohol) (molecular weight of 10 kDa) and 20 mL of anhydrous NMP were combined in a 100 mL round bottom flask. The reaction mixture was heated to 60° C. The resulting solution was subjected to five cycles of azeotropic distillation using anhydrous toluene. After removing the solvent under reduced pressure, the residue was dried under vacuum. Subsequently, the dried polymer was transferred into a 500 mL 3-necked round bottom flask under nitrogen atmosphere to 100 mL of anhydrous NMP was added. To this polymer solution 47.3 g of triphenylphosphine and 29.5 g of N-hydroxyphthalimide was added. The reaction mixture was heated to 45° C. to obtain clear solution. After cooling to −3° C. using a salt/ice bath, 1.6 g of activated molecular sieves (4 Å) was added to the reaction mixture. To this stirred reaction mixture 35.2 mL of diisopropyl azodicarboxylate was added in a drop-wise manner over a period of 30 minutes. The resulting reaction mixture was stirred at −3° C. for 1 hour, slowly warmed to room temperature, and then stirred at room temperature for 48 hours. After removing the molecular sieves by filtration, the solution was precipitated into 2 L of DI water. The solvent was removed and the residue was dissolved in minimal amount of methanol:ethanol mixture (1:1, v/v). The resulting solution was precipitated into 2 L of diethyl ether. After removing the solvent, the residue was dissolved in minimal amount of chloroform and precipitated from 2 L of diethyl ether. After removing the solvent, the precipitate was dried under reduced pressure, yielding 3.8 g of the product.

The dried residue and 40 mL of dioxane:methanol (2:1) were combined in a 250 mL 3-necked round bottom flask. The mixture was stirred until a clear solution was obtained. To this polymer solution was added 8.9 mL of hydrazine monohydrate and the reaction mixture was stirred at reflux temperature for 3 hours. After removing the solvent under reduced pressure, the residue was dispersed in 100 mL of deionized water. The pH of the dispersion was adjusted to 2.0 with concentrated HCl and the resulting reaction mixture was refluxed for 15 minutes. To this reaction mixture 100 mL of DI water was added and the resulting dispersion was refluxed for 45 minutes. After cooling to room temperature, the reaction mixture was filtered and the filtrate was dialyzed against DI water using a 2 kDa dialysis membrane for 72 hours. The dialyzed solution was lyophilized yielding 0.84 g of the desired product as an off white solid.

### Example 3-16-2: Glyoxal Crosslinking of poly(O-vinyl hydroxylamine-co-vinyl alcohol)

0.31 g of Poly(O-vinyl hydroxylamine-co-vinyl alcohol) (Example 3-16-1) and 1.8 mL of DI water were combined in a 10 mL glass vial. After a clear solution was obtained, 8 microliter of 40% aqueous glyoxal was added. The reaction mixture was stirred for 5 minutes to form a soft gel. The gel particles were broken and transferred into a 100 mL round bottom flask with 20 mL of DI water. 63 mg of sodium cyanoborohydride was added to the suspension and the resulting reaction mixture was stirred at 45° C. for 48 hours. After cooling to room temperature, the reaction mixture was filtered. The residue was dispersed in 10 mL of DI water and the pH of the dispersion was brought to 2.0 by slow addition of 1 M HCl. After stirring for 20 minute, the pH was raised to 8.0 by slow addition of 1 M sodium hydroxide. After filtration, the residue was dispersed in 30 mL of DI water and stirred for 30 minutes. The polymer was filtered and this process of water treatment was repeated twice. The residue was finally dialyzed against DI water for 72 hours using an 8 kDa dialysis membrane. The dialyzed polymer gel was lyophilized, yielding 0.12 g of the polymer.

### Example 3-17: Synthesis of epichlorohydrin Crosslinked poly(2-(aminooxy)-N-vinylacetamide-co-vinylamine)

0.15 g of 10 mol % epichlorohydrin crosslinked high molecular weight poly(vinylamine) (Example 3-4) and 15 mL of deionized water were combined in a 100 mL 3-necked round bottom flask. 1.71 g of N-hydroxysuccinimde ester of t-Boc protected aminooxy glycine dissolved in 10.0 mL of dimethylsulfoxide was added to the suspension. The resulting reaction mixture was stirred at 60° C. for 2 hours. 1.65 g of N-hydroxysuccinimde ester of t-Boc protected aminooxy glycine in 2 mL of dimethylsulfoxide was subsequently added and the reaction mixture was stirred at 60° C. for additional 6 hours. The pH of the reaction mixture was maintained at 10.0 with the occasional addition of 1 M NaOH throughout the process. After filtration, the gel particles were suspended in 25 mL of methanol and stirred for 30 minutes. The polymer was filtered and the filtered gel was subjected to the methanol treatment process two more times. Subsequently, the gel was dispersed in 25 mL of DI water, stirred for 30 minutes and filtered. After repeating the water treatment process two more times, the filtered polymer was lyophilized to dryness, yielding 176 mg of the polymer gel.

The polymer gel was combined with 10 mL DI water in a 100 mL 3-necked round bottom flask. While stirring, 4 mL of 1 M HCl was added and the reaction mixture was stirred at 40° C. for 2 hours. 2 mL of 1 M HCl was subsequently added and the reaction mixture was stirred at 40° C. for additional 16 hours. After cooling to room temperature, the pH of the suspension was adjusted to 5.8 by the addition of 1 M NaOH. The polymer gel was filtered and the filtered gel was suspended in 25 mL of DI water. The suspension was stirred for 30 minutes and filtered. After repeating the water treatment process three times, the filtered gel was lyophilized, yielding 125 mg of the desired product.

### Example 3-18: Synthesis of Epichlorohydrin Crosslinked Poly(vinylamine-co-vinylguanidine)

### Example 3-18-1: Synthesis of Epichlorohydrin Crosslinked Poly(vinylamine)

In a 50 mL beaker were taken 2 g of poly(vinylamine) and 18.00 mL of deionized water. The reaction mixture was stirred at room temperature until a clear solution was obtained. To this solution was added 0.36 mL of epichlorohydrin. The resulting reaction mixture was stirred until a gel was formed (after approximately 22 minutes). The stirring was discontinued and the gel was allowed to stand at room temperature for 48 hours. Subsequently the gel was broken into smaller pieces, suspended in 400 mL of deionized water, stirred for 30 minutes, and filtered. After repeating this washing process two more times, the filtered gel was lyophilized yielding 2.2 g of the polymer as an off white solid.

### Example 3-18-2: Synthesis of Epichlorohydrin Crosslinked Poly(vinylamine-co-vinylguanidine)

In a 250 mL three-necked round bottom flask were taken 0.5 g of epichlorohydrin crosslinked poly(vinylamine) (Example 3-18-1) and 50 mL of deionized water. The pH of the suspension was adjusted to 10.55 by adding appropriate amount of 50% (w/w) aqueous NaOH and stirred under nitrogen. In a 100 mL round bottomed flask were taken 6.97 g of pyrazole carboxamidine hydrochloride and 25 mL of deionized water. The pH of the solution was adjusted to 6.85 by adding appropriate amount of 1M NaOH. The resulting solution was added to the polymer suspension and the pH was adjusted to 10.60 by adding appropriate amount of 50% aqueous NaOH. The resulting reaction mixture was stirred under nitrogen at 60° C. for 40 hours. After cooling to room temperature, the suspension was filtered, dispersed in 100 mL of deionized water, stirred for 30 minutes, and filtered. After repeating washing process four more times, the filtered gel was lyophilized yielding 0.52 g of the polymer as an off white solid.

### Example 3-1: Synthesis of 5 mol % Epichlorohydrin Crosslinked Low Molecular Weight Poly(vinylamine)

### Example 3-19: Synthesis of poly(3,4-diaminostyrene-co-divinyl benzene) (95:5)

### Example 3-19-1: Synthesis of divinyl benzene Crosslinked 3,4-diaminostyrene Polymer

All manipulations were carried out by keeping exposure to light at the minimum. 1.0 g of t-Boc protected 3,4-diaminostyrene (Example 3-12-3), 19.5 mg of 1,4-divinylbenzene, 2 g of toluene, and 10 mg of AIBN were combined in a 10 mL glass vial. After flushing the solution with a slow stream of nitrogen for 15 minutes, the reaction mixture was kept at 80° C. for 18 hours, yielding a swollen gel. The gel was treated with 6 mL of methanol followed by 6 mL of 3 M methanolic HCl. The resulting reaction mixture was stirred at room temperature for 18 hours, and then stirred at 45° C. for 3 hours. After removing the solvent, the residue was treated with 10 mL of 1.2 M HCl and dialyzed against DI water using a 12 kDa dialysis membrane for 48 hours. The gel was subsequently lyophilized, yielding 0.4 g of the desired product.

1. A method of binding AGE precursors in a mammal comprising administering to the mammal a compound, wherein the compound comprises the structure of Formula I: 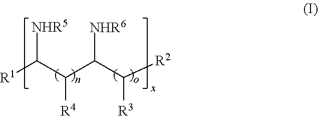
wherein:
n is 0, 1, or 2;
o is 0, 1, or 2;
x is an integer from 2 to 25,000;
R¹ and R² are each independently a pharmaceutically acceptable end group, a polymer, or —R^{x}-polymer,
wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer,
wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

2. The method of claim 1, wherein R¹ and R² are each independently:
H,
a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a guanidino group represented by Formula (A) 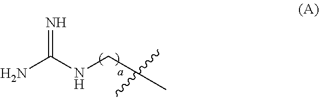
wherein a is an integer from 0 to 25,
a guanidinium chloride group represented by Formula (B), 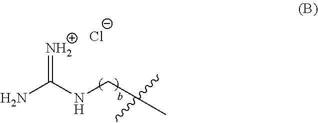
wherein b is an integer from 0 to 25,
a guanidinobenzene group represented by Formula (C), 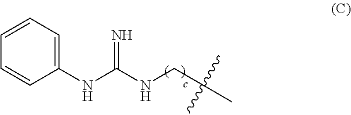
wherein c is an integer from 0 to 25,
a dihydroxy group, represented by Formula (D), 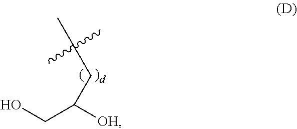
wherein d is an integer from 0 to 25, or
a polyethylene glycol group, represented by Formula (E) 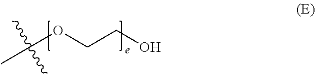
wherein e is an integer from 1 to 400.

3. The method of claim 1, wherein n is 0.

4. The method of claim 1, wherein n is 1.

5. The method of claim 1, wherein n is 2.

6. The method of claim 1, wherein o is 0.

7. The method of claim 1, wherein o is 1.

8. The method of claim 1, wherein o is 2.

9. The method of claim 1, wherein n is 0 and o is 0.

10. The method of claim 1, wherein n is 1 and o is 1.

11. The method of claim 1, wherein the compound is a polymer.

12. The method of claim 11, wherein the polymer is cross-linked.

13. The method of claim 12, wherein the polymer is cross-linked with epichlorohydrin.

14. The method of claim 11, wherein the polymer is a co-polymer.

15. The method of claim 14, wherein the co-polymer is cross-linked.

16. The method of claim 15, wherein the co-polymer is cross-linked with epichlorohydrin.

17. The method of claim 1, wherein R¹ and R² are each independently H or (C₁-C₁₀)alkyl.

18. The method of claim 16, wherein R¹ and R² are each independently H or —CH₃.

19. The method of claim 18, wherein R₁ and R₂ are each H.

20. The method of claim 1, wherein R³ and R⁴ are each independently H or (C₁-C₁₀)alkyl.

21. The method of claim 20, wherein R³ and R⁴ are each independently H or —CH₃.

22. The method of claim 21, wherein R³ and R⁴ are H.

23. The method of claim 1, wherein R⁵ and R⁶ are each independently H or (C₁-C₁₀)alkyl.

24. The method of claim 23, wherein R⁵ and R⁶ are each independently H or —CH₃.

25. The method of claim 24, wherein R⁵ and R⁶ are H.

26. The method of claim 1, wherein R⁵ and R⁶ are taken together with the nitrogens to which they are attached to form a 6 to 20 member ring.

27. The method of claim 26, wherein R⁵ and R⁶ are taken together with the nitrogens to which they are attached to form a 14 member ring.

28. The method of claim 1, wherein:
n is 1;
o is 1;
R¹ and R² are each independently a pharmaceutically acceptable end group;
R³ and R⁴ are each H; and
R⁵ and R⁶ are each H.

29. A method of binding AGE precursors in a mammal comprising administering to the mammal a compound, wherein the compound comprises the structure of Formula I-A: 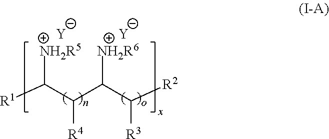
wherein:
n is 0, 1, or 2;
o is 0, 1, or 2;
x is an integer from 2 to 25,000;
Y⁻ is each independently a pharmaceutically acceptable anion;
R¹ and R² are each independently a pharmaceutically acceptable end group, a polymer, or —R^{x}-polymer,
wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide;
R³ and R⁴ are each independently H, a polymer, or —R^{x}-polymer,
wherein R^{x} is selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₁-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide,
or if n is 0 R⁴ is absent, and if o is 0 R³ is absent; and
R⁵ and R⁶ are each independently H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, —NH₂, —NH(C₁-C₁₀)alkyl, —N[(C₁-C₁₀)alkyl]₂ or
R⁵ and R⁶ are taken together with the nitrogens to which they are attached to form a 6 to 20 member ring.

30. The method of claim 29, wherein R¹ and R² are each independently:
H,
a group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a guanidino group represented by Formula (A) 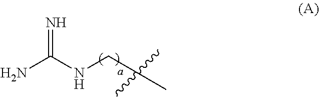
wherein a is an integer from 0 to 25,
a guanidinium chloride group represented by Formula (B), 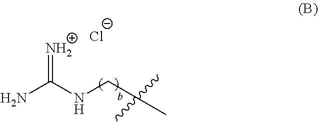
wherein b is an integer from 0 to 25,
a guanidinobenzene group represented by Formula (C), 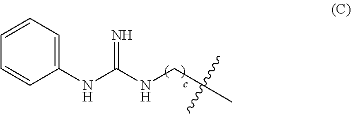
wherein c is an integer from 0 to 25,
a dihydroxy group, represented by Formula (D), 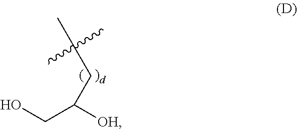
wherein d is an integer from 0 to 25, or
a polyethylene glycol group, represented by Formula (E) 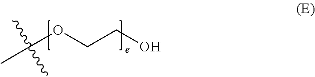
wherein e is an integer from 1 to 400.

31. The method of claim 29, wherein Y⁻ is independently carbonate, bicarbonate, or chloride.

32. The method of claim 31, wherein Y⁻ is independently carbonate or bicarbonate.

33. The method of claim 31, wherein Y⁻ is chloride.

34. The method of claim 29, wherein n is 0.

35. The method of claim 29, wherein n is 1.

36. The method of claim 29, wherein n is 2.

37. The method of claim 29, wherein o is 0.

38. The method of claim 29, wherein o is 1.

39. The method of claim 29, wherein o is 2.

40. The method of claim 29, wherein n is 0 and o is 0.

41. The method of claim 29, wherein n is 1 and o is 1.

42. The method of claim 29, wherein the compound is a polymer.

43. The method of claim 42, wherein the polymer is cross-linked.

44. The method of claim 43, wherein the polymer is cross-linked with epichlorohydrin.

45. The method of claim 42, wherein the polymer is a co-polymer.

46. The method of claim 45, wherein the co-polymer is cross-linked.

47. The method of claim 46, wherein the co-polymer is cross-linked with epichlorohydrin.

48. The method of claim 29, wherein R¹ and R² are each independently H or (C₁-C₁₀)alkyl.

49. The method of claim 47, wherein R¹ and R² are each independently H or —CH₃.

50. The method of claim 49, wherein R¹ and R² are each H.

51. The method of claim 29, wherein R³ and R⁴ are each independently H or (C₁-C₁₀)alkyl.

52. The method of claim 51, wherein R³ and R⁴ are each independently H or —CH₃.

53. The method of claim 52, wherein R₃ and R₄ are H.

54. The method of claim 29, wherein R⁵ and R⁶ are each independently H or (C₁-C₁₀)alkyl.

55. The method of claim 54, wherein R⁵ and R⁶ are each independently H or —CH₃.

56. The method of claim 55, wherein R⁵ and R⁶ are H.

57. The method of claim 29, wherein R⁵ and R⁶ are taken together with the nitrogens to which they are attached to form a 6 to 20 member ring.

58. The method of claim 57, wherein R⁵ and R⁶ are taken together with the nitrogens to which they are attached to form a 14 member ring.

59. The method of claim 29, wherein:
n is 1;
o is 1;
R¹ and R² are each independently a pharmaceutically acceptable end group; and
R³ and R⁴ are each independently H.

60.-122. (canceled)
